(19) United States
(12) Patent Application Publication
(10) Pub. No.: US 2024/0391927 A1
(43) Pub. Date: Nov. 28, 2024

(54) UNIFIED SYNTHETIC ENTRY TO AZASPIRO 3.N ALKANES

(71) Applicant: The Florida State University Research Foundation, Inc., Tallahassee, FL (US)

(72) Inventors: James H. FREDERICH, Tallahassee, FL (US); Tyler D. WEINHOLD, Tallahassee, FL (US)

(21) Appl. No.: 18/664,869

(22) Filed: May 15, 2024

Related U.S. Application Data
(60) Provisional application No. 63/466,448, filed on May 15, 2023.

(51) Int. Cl. C07D 487/10; C07D 205/04; C07D 205/12; C07D 401/04

(52) U.S. Cl. CPC C07D 487/10; C07D 205/04; C07D 205/12; C07D 401/04

(57) ABSTRACT

Described herein are processes for preparing compounds of Formula I, Formula IIa, Formula IIb, Formula IIc, and Formula IId.

### CROSS REFERENCE TO RELATED APPLICATIONS

This application claims the benefit of priority to U.S. Provisional Application 63/466,448, filed May 15, 2023, which is incorporated by reference herein in its entirety.

### ACKNOWLEDGEMENT OF GOVERNMENT SUPPORT

This invention was made with government support under Grant No. R01GM125926 awarded by the National Institutes of Health. The government has certain rights in the invention.

### BACKGROUND

The synthesis of all-carbon quaternary centers is an enduring challenge in organic chemistry (Quasdorf, K. W., et al., Nature. 2014, 516, 181; Susse, L., et al., Chem. Rev. 2021, 121, 4084). This functional group is prevalent in bioactive natural products (Li, C., et al., Nat. Prod. Rep. 2020, 37, 276), and recently, there is a growing demand for novel quaternary carbon building blocks for medicinal chemistry (Talele, T. T., et al., J. Med. Chem. 2020, 63, 13291). Introduced in this context by Muller and Carreira (Bukhard, J. A., et al., Angew. Chem., Int. Ed. 2010, 49, 3524 (“Bukhard, et al., 2010”); Zhou, J., et al., Org. Lett. 2020, 22, 4413 (“Zhou, J., et al., 2020”)), nitrogen-containing spiro[3.n]alkanes featuring a quaternary center have generated interest across academia and industry (Zhou, J., et al., 2020; Hiesinger, K., et al., J. Med. Chem. 2021, 64, 150). This structural class includes the 2,6-diazaspiro[3.3]heptane ring system, which has gained attention as a isostere for piperazine (Litherland, A., et al., Part I. Preparation. J. Chem. Soc. 1938, 1588). Incorporation of this heterocycle into drug-like small molecules has been shown to increase conformational rigidity, improve solubility, and importantly, occupy chemical space that is inaccessible any other way (Bukhard, et al., 2010; Degorce, S. L., et al., ACS Med. Chem. Lett. 2019, 10, 1198). Exploiting these features, Mach and co-workers demonstrated that replacing the piperazine ring of olaparib (1) with a 2,6-diazaspiro[3.3]heptane (i.e., 2) enhanced target selectivity and reduced off-mechanism cytotoxicity in human cell culture (Reilly, S. W., et al., J. Med. Chem. 2018, 61, 5367). Together, these studies highlight the significant potential of azaspiro[3.n]alkanes and their congeners as three-dimensional inputs for drug discovery (Lovering, F., et al., J. Med. Chem. 2009, 52, 6752; Klein, H. F., et al., Drug Discov. Today 2022, 27, 2484).

While unassuming at first glance, azaspirocycles are intricate structures that continue to inspire new advances in heterocyclic chemistry (Carreira, E. M., et al., Chem. Rev. 2014, 114, 8257; Grygorenko, O. O., et al., Eur. J. Org. Chem. 2021, 6478; Tyler, J. L., et al., Angew. Chem., Int. Ed. 2021, 60, 11824; Murray, P. R. D., et al., J. Am. Chem. Soc. 2021, 143, 4055; Novoa, L., et al., Angew. Chem., Int. Ed. 2021, 60, 11763). Nevertheless, as a consequence of the quaternary center embedded within azaspiro[3.n]alkanes, this substructure remains laborious to prepare. For example, monoprotected 2,6-diazaspiro[3.3]heptanes require an 8-step synthesis that begins from a quaternary carbon fragment (Burkhard, J., et al., Org. Lett. 2008, 10, 3525; Hamza, D., et al., Synlett. 2007, 16, 2584; Meyers, M. J., et al., Org. Lett. 2009, 11, 3523; Kirichok, A. A., et al., Angew. Chem., Int. Ed. 2017, 56, 8865). In contrast, the related 2,6-diazaspiro [3.4]octane and -[3.5]nonane motifs are prepared along a 6-step route that leverages enolate acylation to build the quaternary center (Orain, D., et al., Synlett 2015, 26, 1815).

While these distinct solutions can support discovery research, a modular and more concise entry point to azaspiro[3.n]alkanes is required to streamline their integration into medicinal chemistry programs. The compositions and methods disclosed herein address these and other needs.

### SUMMARY

Provided herein are processes and intermediates useful for the preparation of compounds of Formula I, IIa, IIb, IIc, and IId.

In some embodiments, described herein is a process for preparing a compound of Formula I

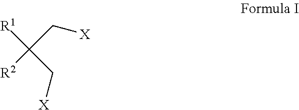

comprising:
(a) treating a compound of formula 10

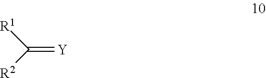

with a compound of formula 11

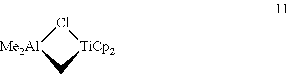

to form a first intermediate and treating the first intermediate with M₂ to form the compound of Formula I;
wherein:
X is a halogen;
Y is O or CH₂;
M is Cl, Br, or I; and
R¹ and R² are each independently selected from hydrogen, substituted or unsubstituted alkyl, cycloalkyl, alkoxy, aryl or heteroaryl, alkylaryl, ether, or R¹ and R², taken together with the atom to which they are directly attached form a four or more membered ring; and at least one of R¹ or R² are substituted or unsubstituted alkyl, cycloalkyl, alkoxy, aryl or heteroaryl, alkylaryl, or ether.

In some embodiments, the first intermediate can be defined by the structure below:

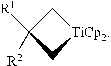

In some embodiments, described herein is a process for preparing a compound of Formula IIa

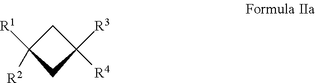

comprising:
(a) treating a compound of Formula I

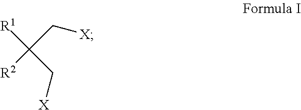

with

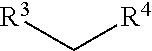

in the presence of a base to form a compound of Formula IIa;
wherein:
X is a halogen;
R¹ and R² are each independently selected from hydrogen, substituted or unsubstituted alkyl, cycloalkyl, alkoxy, aryl or heteroaryl, alkylaryl, ether, or R¹ and R², taken together with the atom to which they are directly attached form a four or more membered ring; and at least one of R¹ or R² are substituted or unsubstituted alkyl, cycloalkyl, alkoxy, aryl or heteroaryl, alkylaryl, or ether; and
R³ and R⁴ are ester.

In some embodiments, described herein is a process for preparing a compound of Formula IIb

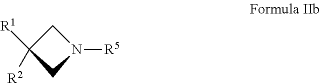

comprising:
(a) treating a compound of Formula I

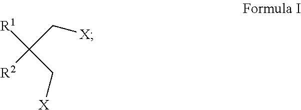

with R⁵—NH₂ in the presence of a base to form a compound of Formula IIb;
wherein:
X is a halogen;
R¹ and R² are each independently selected from hydrogen, substituted or unsubstituted alkyl, cycloalkyl, alkoxy, aryl or heteroaryl, alkylaryl, ether, or R¹ and R², taken together with the atom to which they are directly attached form a four or more membered ring; and at least one of R¹ or R² are substituted or unsubstituted alkyl, cycloalkyl, alkoxy, aryl or heteroaryl, alkylaryl, or ether; and
R⁵ is substituted or unsubstituted alkenyl, alkylaryl, aryl, or heteroaryl.

In some embodiments, described herein is a process for preparing a compound of Formula IIc

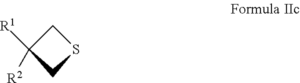

comprising:
(a) treating a compound of Formula I

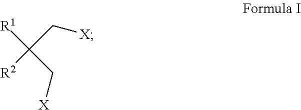

with Na₂S to form a compound of Formula IIc;
wherein:
X is a halogen;
R¹ and R² are each independently selected from hydrogen, substituted or unsubstituted alkyl, cycloalkyl, alkoxy, aryl or heteroaryl, alkylaryl, ether, or R¹ and R², taken together with the atom to which they are directly attached form a four or more membered ring; and at least one of R¹ or R² are substituted or unsubstituted alkyl, cycloalkyl, alkoxy, aryl or heteroaryl, alkylaryl, or ether.

In some embodiments, described herein is a process for preparing a compound of Formula IId

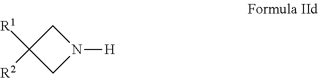

comprising:
(a) preparing a compound of Formula IIa described herein; and
(b) hydrogenolysis of compound of Formula IIa to form a compound of Formula IId;
wherein:
R¹ and R² are each independently selected from hydrogen, substituted or unsubstituted alkyl, cycloalkyl, alkoxy, aryl or heteroaryl, alkylaryl, ether, or R¹ and R², taken together with the atom to which they are directly attached form a four or more membered ring; and at least one of R¹ or R² are substituted or unsubstituted alkyl, cycloalkyl, alkoxy, aryl or heteroaryl, alkylaryl, or ether.

In some embodiments, the process further can include isolating the compound of Formula I. In some embodiments, the process further can include isolating the compound of Formula IIa. In some embodiments, the process further can include isolating the compound of Formula IIb. In some embodiments, the process further can include isolating the compound of Formula IIc. In some embodiments, the process further can include isolating the compound of Formula IId.

### BRIEF DESCRIPTION OF THE FIGURES

FIG. 1 shows an exemplary synthetic scheme.

FIG. 2 shows ¹H NMR of the unpurified reaction mixture comparing Br₂ to NBS.

FIG. 3 shows observation of 14, d-14, and S9 by ¹H NMR spectroscopy.

FIG. 4 is ¹H NMR spectra for stability experiment. A solution of 16 (in CDCl₃) was maintained at room temperature under ambient conditions and monitored periodically via ¹H NMR slow, non-specific decomposition was notice after 13 days.

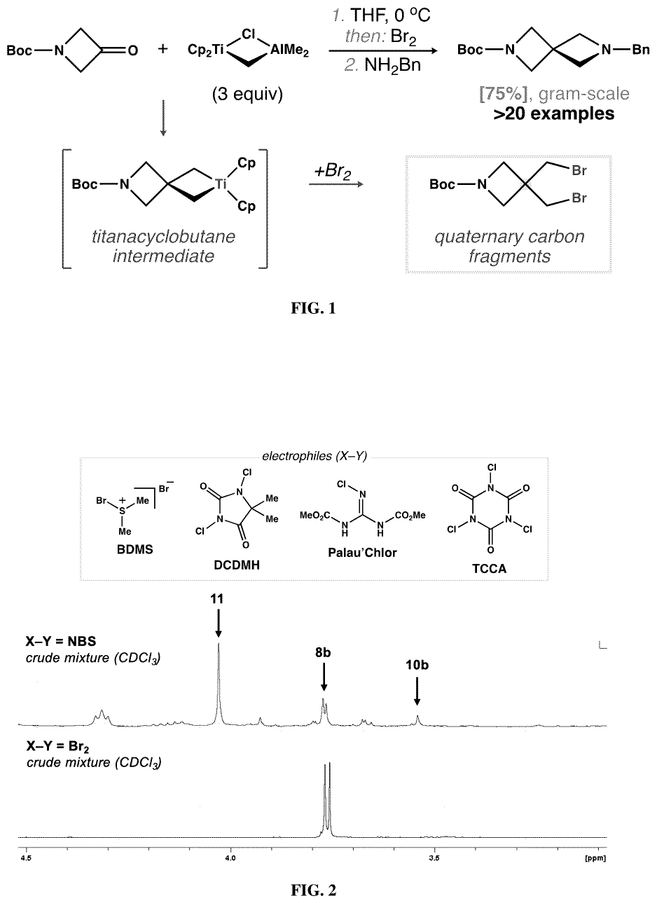

### DETAILED DESCRIPTION

Before the present methods and systems are disclosed and described, it is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes¬from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

Compounds disclosed herein may be provided in the form of physiologically acceptable salts. Examples of such salts are acid addition salts formed with inorganic acids, for example, hydrochloric, hydrobromic, sulfuric, phosphoric, and nitric acids and the like; salts formed with organic acids such as acetic, oxalic, tartaric, succinic, maleic, fumaric, gluconic, citric, malic, methanesulfonic, p-toluenesulfonic, napthalenesulfonic, and polygalacturonic acids, and the like; salts formed from elemental anions such as chloride, bromide, and iodide; salts formed from metal hydroxides, for example, sodium hydroxide, potassium hydroxide, calcium hydroxide, lithium hydroxide, and magnesium hydroxide; salts formed from metal carbonates, for example, sodium carbonate, potassium carbonate, calcium carbonate, and magnesium carbonate; salts formed from metal bicarbonates, for example, sodium bicarbonate and potassium bicarbonate; salts formed from metal sulfates, for example, sodium sulfate and potassium sulfate; and salts formed from metal nitrates, for example, sodium nitrate and potassium nitrate.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions, Wiley Interscience, New York, 1981; Wilen et al., Tetrahedron 33:2725 (1977); Eliel, E. L. Stereochemistry of Carbon Compounds, McGraw-Hill, NY, 1962; and Wilen, S. H., Tables of Resolving Agents and Optical Resolutions p. 268, E. L. Eliel, Ed., Univ. of Notre Dame Press, Notre Dame, IN 1972. The invention additionally encompasses compounds as individual isomers substantially free of other isomers, and alternatively, as mixtures of various isomers.

When a range of values is listed, it is intended to encompass each value and sub-range within the range. For example, “C₁₋₆ alkyl” is intended to encompass C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “alkyl” refers to a radical of a straight-chain or branched hydrocarbon group having a specified range of carbon atoms (e.g., a “C₁₋₁₆ alkyl” can have from 1 to 16 carbon atoms). In some embodiments, an alkyl group has 1 to 9 carbon atoms (“C₁₋₉ alkyl”).

An alkyl group can be saturated or unsaturated, i.e., an alkenyl or alkynyl group as defined herein. Unless specified to the contrary, an “alkyl” group includes both saturated alkyl groups and unsaturated alkyl groups.

In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, iso-butyl), pentyl (C₅) (e.g., n-pentyl, 3-pentanyl, amyl, neopentyl, 3-methyl-2-butanyl, tertiary amyl), and hexyl (C₆) (e.g., n-hexyl). Additional examples of alkyl groups include n-heptyl (C₇), n-octyl (C₈), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₀ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu), unsubstituted isobutyl (i-Bu)). In certain embodiments, the alkyl group is a substituted C₁₋₁₀ alkyl (such as substituted C₁₋₆ alkyl, e.g., —CF₃, Bn).

The term “haloalkyl” is a substituted alkyl group, wherein one or more of the hydrogen atoms are independently replaced by a halogen, e.g., fluoro, bromo, chloro, or iodo. In some embodiments, the haloalkyl moiety has 1 to 8 carbon atoms (“C₁₋₈ haloalkyl”). In some embodiments, the haloalkyl moiety has 1 to 6 carbon atoms (“C₁₋₆ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). Examples of haloalkyl groups include —CHF₂, —CH₂F, —CF₃, —CH₂CF₃, —CF₂CF₃, —CF₂CF₂CF₃, —CCl₃, —CFCl₂, —CF₂Cl, and the like.

The term “hydroxyalkyl” is a substituted alkyl group, wherein one or more of the hydrogen atoms are independently replaced by a hydroxyl. In some embodiments, the hydroxyalkyl moiety has 1 to 8 carbon atoms (“C₁₋₈ hydroxyalkyl”). In some embodiments, the hydroxyalkyl moiety has 1 to 6 carbon atoms (“C₁₋₆ hydroxyalkyl”). In some embodiments, the hydroxyalkyl moiety has 1 to 4 carbon atoms (“C₁₋₄ hydroxyalkyl”). In some embodiments, the hydroxyalkyl moiety has 1 to 3 carbon atoms (“C₁₋₃ hydroxyalkyl”). In some embodiments, the hydroxyalkyl moiety has 1 to 2 carbon atoms (“C₁₋₂ hydroxyalkyl”).

The term “alkoxy” refers to an alkyl group, as defined herein, appended through an oxygen atom. In some embodiments, the alkoxy moiety has 1 to 8 carbon atoms (“C₁₋₈ alkoxy”). In some embodiments, the alkoxy moiety has 1 to 6 carbon atoms (“C₁₋₆ alkoxy”). In some embodiments, the alkoxy moiety has 1 to 4 carbon atoms (“C₁₋₄ alkoxy”). In some embodiments, the alkoxy moiety has 1 to 3 carbon atoms (“C₁₋₃ alkoxy”). In some embodiments, the alkoxy moiety has 1 to 2 carbon atoms (“C₁₋₂ alkoxy”). Representative examples of alkoxy include, but are not limited to, methoxy, ethoxy, propoxy, 2-propoxy, butoxy and tert-butoxy.

The term “haloalkoxy” refers to a haloalkyl group, as defined herein, appended through an oxygen atom. In some embodiments, the alkoxy moiety has 1 to 8 carbon atoms (“C₁₋₈ haloalkoxy”). In some embodiments, the alkoxy moiety has 1 to 6 carbon atoms (“C₁₋₆ haloalkoxy”). In some embodiments, the alkoxy moiety has 1 to 4 carbon atoms (“C₁₋₄ haloalkoxy”). In some embodiments, the alkoxy moiety has 1 to 3 carbon atoms (“C₁₋₃ haloalkoxy”). In some embodiments, the alkoxy moiety has 1 to 2 carbon atoms (“C₁₋₂ haloalkoxy”). Representative examples of haloalkoxy include, but are not limited to, difluoromethoxy, trifluoromethoxy, and 2,2,2-trifluoroethoxy.

The term “alkoxyalkyl” is a substituted alkyl group, wherein one or more of the hydrogen atoms are independently replaced by an alkoxy group, as defined herein. In some embodiments, the alkoxyalkyl moiety has 1 to 8 carbon atoms (“C₁₋₈ alkoxyalkyl”). In some embodiments, the alkoxyalkyl moiety has 1 to 6 carbon atoms (“C₁₋₆ alkoxyalkyl”). In some embodiments, the alkoxyalkyl moiety has 1 to 4 carbon atoms (“C₁₋₄ alkoxyalkyl”). In some embodiments, the alkoxyalkyl moiety has 1 to 3 carbon atoms (“C₁₋₃ alkoxyalkyl”). In some embodiments, the alkoxyalkyl moiety has 1 to 2 carbon atoms (“C₁₋₂ alkoxyalkyl”). By way of example, a C₃alkoxyC₃alkyl group includes, but is not limited to, the groups having the formula:

—CH₂CH₂CH₂OCH₂CH₂CH₃,—CH₂CH₂CH₂OCH(CH₃)₂,—CH(CH₃)CH₂OCH(CH₃)₂,

The term “heteroalkyl” refers to an alkyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. By way of example, a heteroC₁₋₆alkyl (which may also be designated a C₁₋₆heteroalkyl) group includes, but is not limited to, the following structures:

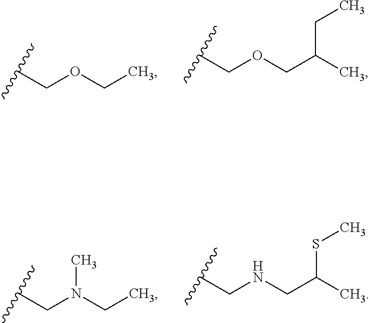

The term “heteroalkyl” preceded by a separate heteroatom refers to a heteroalkyl group bonded through the specified heteroatom. By way of example, a OC₁₋₆heteroalkyl group includes, but it not limited to, the following structures:

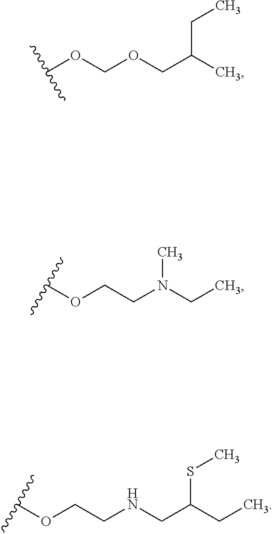

In certain embodiments, a heteroalkyl group refers to a saturated group having from 1 to 20 carbon atoms and 1 or more heteroatoms within the parent chain (“heteroC₁₋₂₀ alkyl”). In some embodiments, a heteroalkyl group is a saturated group having 1 to 18 carbon atoms and 1 or more heteroatoms within the parent chain (“heteroC₁₋₁₈ alkyl”). In some embodiments, a heteroalkyl group is a saturated group having 1 to 16 carbon atoms and/or more heteroatoms within the parent chain (“heteroC₁₋₆ alkyl”). In some embodiments, a heteroalkyl group is a saturated group having 1 to 14 carbon atoms and 1 or more heteroatoms within the parent chain (“heteroC₁₋₁₄ alkyl”). In some embodiments, a heteroalkyl group is a saturated group having 1 to 12 carbon atoms and 1 or more heteroatoms within the parent chain (“heteroC₁₋₁₂ alkyl”). In some embodiments, a heteroalkyl group is a saturated group having 1 to 10 carbon atoms and 1 or more heteroatoms within the parent chain (“heteroC₁₋₁₀ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃alkyl”). In some embodiments, a heteroalkyl group is a saturated group having 1 to 2 carbon atoms and 1 heteroatom within the parent chain (“heteroC₁₋₂alkyl”). In some embodiments, a heteroalkyl group is a saturated group having 1 carbon atom and 1 heteroatom (“heteroC₁alkyl”). In some embodiments, the heteroalkyl group defined herein is a partially unsaturated group having 1 or more heteroatoms within the parent chain and at least one unsaturated carbon, such as a carbonyl group. For example, a heteroalkyl group may comprise an amide or ester functionality in its parent chain such that one or more carbon atoms are unsaturated carbonyl groups. Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₂₀alkyl. In certain embodiments, the heteroalkyl group is an unsubstituted heteroC₁₋₁₀alkyl. In certain embodiments, the heteroalkyl group is a substituted heteroC₁₋₂₀alkyl. In certain embodiments, the heteroalkyl group is an unsubstituted heteroC₁₋₁₀alkyl.

The term “alkenyl” refers to a radical of a straight-chain or branched hydrocarbon group having from 2 to 10 carbon atoms and one or more carbon-carbon double bonds (e.g., 1, 2, 3, or 4 double bonds). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆alkenyl”). In some embodiments, an alkenyl group has 2 to 5 carbon atoms (“C₂₋₅ alkenyl”). In some embodiments, an alkenyl group has 2 to 4 carbon atoms (“C₂₋₄alkenyl”). In some embodiments, an alkenyl group has 2 to 3 carbon atoms (“C₂₋₃alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₂₋₁₀ alkenyl. In certain embodiments, the alkenyl group is a substituted C₂₋₁₀ alkenyl. In an alkenyl group, a C═C double bond for which the stereochemistry is not specified (e.g., —CH═CHCH₃ or

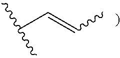

may be an (E)- or (Z)-double bond.

The term “heteroalkenyl” refers to an alkenyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkenyl group refers to a group having from 2 to 10 carbon atoms, at least one double bond, and 1 or more heteroatoms within the parent chain (“heteroC₂₋₁₀alkenyl”). In some embodiments, a heteroalkenyl group has 2 to 9 carbon atoms at least one double bond, and 1 or more heteroatoms within the parent chain (“heteroC₂₋₉alkenyl”).

In some embodiments, a heteroalkenyl group has 2 to 8 carbon atoms, at least one double bond, and 1 or more heteroatoms within the parent chain (“heteroC₂₋₈alkenyl”). In some embodiments, a heteroalkenyl group has 2 to 7 carbon atoms, at least one double bond, and 1 or more heteroatoms within the parent chain (“heteroC₂₋₇alkenyl”). In some embodiments, a heteroalkenyl group has 2 to 6 carbon atoms, at least one double bond, and 1 or more heteroatoms within the parent chain (“heteroC₂₋₆alkenyl”). In some embodiments, a heteroalkenyl group has 2 to 5 carbon atoms, at least one double bond, and 1 or 2 heteroatoms within the parent chain (“heteroC₂₋₅alkenyl”). In some embodiments, a heteroalkenyl group has 2 to 4 carbon atoms, at least one double bond, and 1 or 2 heteroatoms within the parent chain (“heteroC₂₋₄ alkenyl”). In some embodiments, a heteroalkenyl group has 2 to 3 carbon atoms, at least one double bond, and 1 heteroatom within the parent chain (“heteroC₂₋₃alkenyl”). In some embodiments, a heteroalkenyl group has 2 to 6 carbon atoms, at least one double bond, and 1 or 2 heteroatoms within the parent chain (“heteroC₂₋₆alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₁₀alkenyl. In certain embodiments, the heteroalkenyl group is a substituted heteroC₂₋₁₀alkenyl.

The term “alkynyl” refers to a radical of a straight-chain or branched hydrocarbon group having from 2 to 10 carbon atoms and one or more carbon-carbon triple bonds (e.g., 1, 2, 3, or 4 triple bonds) (“C₂₋₁₀alkynyl”). In some embodiments, an alkynyl group has 2 to 9 carbon atoms (“C₂₋₉alkynyl”). In some embodiments, an alkynyl group has 2 to 8 carbon atoms (“C₂₋₈alkynyl”). In some embodiments, an alkynyl group has 2 to 7 carbon atoms (“C₂₋₇alkynyl”). In some embodiments, an alkynyl group has 2 to 6 carbon atoms (“C₂₋₆alkynyl”). In some embodiments, an alkynyl group has 2 to 5 carbon atoms (“C₂₋₅alkynyl”). In some embodiments, an alkynyl group has 2 to 4 carbon atoms (“C₂₋₄alkynyl”). In some embodiments, an alkynyl group has 2 to 3 carbon atoms (“C₂₋₃alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₂₋₁₀ alkynyl. In certain embodiments, the alkynyl group is a substituted C₂₋₁₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkynyl group refers to a group having from 2 to 10 carbon atoms, at least one triple bond, and 1 or more heteroatoms within the parent chain (“heteroC₂₋₁₀alkynyl”). In some embodiments, a heteroalkynyl group has 2 to 9 carbon atoms, at least one triple bond, and 1 or more heteroatoms within the parent chain (“heteroC₂₋₉alkynyl”). In some embodiments, a heteroalkynyl group has 2 to 8 carbon atoms, at least one triple bond, and 1 or more heteroatoms within the parent chain (“heteroC₂₋₈alkynyl”). In some embodiments, a heteroalkynyl group has 2 to 7 carbon atoms, at least one triple bond, and 1 or more heteroatoms within the parent chain (“heteroC₂₋₇alkynyl”). In some embodiments, a heteroalkynyl group has 2 to 6 carbon atoms, at least one triple bond, and 1 or more heteroatoms within the parent chain (“heteroC₂₋₆alkynyl”). In some embodiments, a heteroalkynyl group has 2 to 5 carbon atoms, at least one triple bond, and 1 or 2 heteroatoms within the parent chain (“heteroC₂₋₅alkynyl”). In some embodiments, a heteroalkynyl group has 2 to 4 carbon atoms, at least one triple bond, and 1 or 2 heteroatoms within the parent chain (“heteroC₂₋₄alkynyl”). In some embodiments, a heteroalkynyl group has 2 to 3 carbon atoms, at least one triple bond, and 1 heteroatom within the parent chain (“heteroC₂₋₃alkynyl”). In some embodiments, a heteroalkynyl group has 2 to 6 carbon atoms, at least one triple bond, and 1 or 2 heteroatoms within the parent chain (“heteroC₂-6alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₁₀alkynyl. In certain embodiments, the heteroalkynyl group is a substituted heteroC₂₋₁₀alkynyl.

The term “carbocyclyl,” “cycloalkyl,” or “carbocyclic” refers to a radical of a non-aromatic cyclic hydrocarbon group having from 3 to 14 ring carbon atoms (“C₃₋₁₄carbocyclyl”) and zero heteroatoms in the non-aromatic ring system. In some embodiments, a carbocyclyl group has 3 to 10 ring carbon atoms (“C₃₋₁₀carbocyclyl”). In some embodiments, a carbocyclyl group has 3 to 8 ring carbon atoms (“C₃₋₈carbocyclyl”). In some embodiments, a carbocyclyl group has 3 to 7 ring carbon atoms (“C₃₋₇carbocyclyl”). In some embodiments, a carbocyclyl group has 3 to 6 ring carbon atoms (“C₃₋₆carbocyclyl”). In some embodiments, a carbocyclyl group has 4 to 6 ring carbon atoms (“C₄₋₆carbocyclyl”). In some embodiments, a carbocyclyl group has 5 to 6 ring carbon atoms (“C₅₋₆carbocyclyl”). In some embodiments, a carbocyclyl group has 5 to 10 ring carbon atoms (“C₅₋₁₀carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C), cyclohexenyl (C), cyclohexadienyl (C₆), and the like.

Exemplary C₃₋₈carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄cycloalkyl”). In some embodiments, a cycloalkyl group has 3 to 10 ring carbon atoms (“C₃₋₁₀cycloalkyl”). In some embodiments, a cycloalkyl group has 3 to 8 ring carbon atoms (“C₃₋₈cycloalkyl”). In some embodiments, a cycloalkyl group has 3 to 6 ring carbon atoms (“C₃₋₆cycloalkyl”). In some embodiments, a cycloalkyl group has 4 to 6 ring carbon atoms (“C₄₋₆cycloalkyl”). In some embodiments, a cycloalkyl group has 5 to 6 ring carbon atoms (“C₅₋₆cycloalkyl”). In some embodiments, a cycloalkyl group has 5 to 10 ring carbon atoms (“C₅₋₁₀cycloalkyl”). Examples of C₅₋₆ cycloalkyl groups include cyclopentyl (C₅) and cyclohexyl (C₆). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl.

As used herein, the term “heterocyclyl” refers to an aromatic (also referred to as a heteroaryl), unsaturated, or saturated cyclic hydrocarbon that includes at least one heteroatom in the cycle. For example, the term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include, without limitation, aziridinyl, oxiranyl, and thiiranyl. Exemplary 4-membered heterocyclyl groups containing 1 heteroatom include, without limitation, azetidinyl, oxetanyl, and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, and dioxanyl. Exemplary 6-membered heterocyclyl groups containing 3 heteroatoms include, without limitation, triazinyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₁₋₁₄aryl”). In some embodiments, an aryl group has 6 ring carbon atoms (“C₆aryl”; e.g., phenyl). In some embodiments, an aryl group has 10 ring carbon atoms (“C₁₀aryl”; e.g., naphthyl such as 1-naphthyl and 2-naphthyl). In some embodiments, an aryl group has 14 ring carbon atoms (“C₁₋₄aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄aryl. In certain embodiments, the aryl group is a substituted C₆₋₁₄ aryl.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include, without limitation, pyrrolyl, furanyl, and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl, and phenazinyl.

In general, the inclusion of the prefix “alk” in front of a substituent name indicates there is an alkyl group (as defined herein) connecting the named substituent with the rest of the compound. For example, “alkaryl” (which is a subset of alkyl) refers to an alkyl group substituted by an aryl group, wherein the point of attachment is on the alkyl moiety and “alkheteroaryl” (which is a subset of “alkyl”) refers to an alkyl group substituted by a heteroaryl group, wherein the point of attachment is on the alkyl moiety. The number of carbons atoms may be specified in the alkyl chain, the named substituent, or both. For example, C₁₋₂alkC₆aryl refers to a phenyl ring (which may be substituted) connected via a 1-2 carbon alkylene group.

Affixing the suffix “-ene” to a group indicates the group is a polyvalent moiety, e.g., boned to two or more groups. Alkylene is the polyvalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which may be substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds and includes any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety. The invention is not intended to be limited in any manner by the exemplary substituents described herein.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₃, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃, —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR)₂, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{cc})₂, —B(OR^{cc})₂, —BR^{aa}(OR^{cc}), C₁₋₁₀alkyl, C₁₋₁₀perhaloalkyl, C₂₋₁₀ alkenyl, C₂₋₁₀alkynyl, heteroC₁₋₁₀alkyl, heteroC₂₋₁₀ alkenyl, heteroC₂₋₁₀alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion; or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb} or ═NOR^{cc}; each instance of R^{aa} is, independently, selected from C₁₋₁₀alkyl, C₁₋₁₀perhaloalkyl, C₂₋₁₀alkenyl, C₂₋₁₀ alkynyl, heteroC₁₋₁₀alkyl, heteroC₂₋₁₀ alkenyl, heteroC₂₋₁₀alkynyl, C₃₋₁₀ carbocyclyl, 3-14 membered heterocyclyl, C₆₋₁₄aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; each instance of R^{bb} is, independently, selected from hydrogen, —OH, —OR^{aa}, —N(R^{cc})₂, —CN, —C(═O)Ra, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₁₀ alkyl, C₁₋₁₀perhaloalkyl, C₂₋₁₀alkenyl, C₂₋₁₀alkynyl, heteroC₁₋₁₀alkyl, heteroC₂₋₁₀alkenyl, heteroC₂₋₁₀alkynyl, C₃₋₁₀carbocyclyl, 3-14 membered heterocyclyl, C₆₋₁₄aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion; each instance of R^{cc} is, independently, selected from hydrogen, C₁₋₁₀alkyl, C₁₋₁₀perhaloalkyl, C₂₋₁₀alkenyl, C₂₋₁₀alkynyl, heteroC₁₋₁₀ alkyl, heteroC₂₋₁₀alkenyl, heteroC₂₋₁₀alkynyl, C₃₋₁₀carbocyclyl, 3-14 membered heterocyclyl, C₆₋₁₄aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂-6 alkynyl, heteroC₁₋₆ alkyl, heteroC₂₋₆ alkenyl, heteroC₂₋₆ alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; wherein X⁻ is a counterion; each instance of R^{ee} is, independently, selected from C₁₋₆alkyl, C₁₋₆perhaloalkyl, C₂-alkenyl, C₂₋₆alkynyl, heteroC₁₋₆ alkyl, heteroC₂₋₆alkenyl, heteroC₂₋₆alkynyl, C₃₋₁₀carbocyclyl, C₆₋₁₀aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; each instance of R^{ff} is, independently, selected from hydrogen, C₁₋₆ alkyl, C₁₋₆ perhaloalkyl, C₂₋₆ alkenyl, C₂-6 alkynyl, heteroC₁₋₆ alkyl, heteroC₂₋₆ alkenyl, heteroC₂₋₆ alkynyl, C₃₋₁₀ carbocyclyl, 3-10 membered heterocyclyl, C₆₋₁₀ aryl and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)+X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(═NH)NH(C₁₋₆ alkyl), —OC(═NH)NH₂, —NHC(═NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂(C₁₋₆ alkyl), —SO₂O(C₁₋₆ alkyl), —OSO₂(C₁₋₆ alkyl), —SO(C₁₋₆ alkyl), —Si(C₁₋₆ alkyl)₃, —OSi(C₁₋₆ alkyl)₃, —C(═S)N(C₁₋₆ alkyl)₂, —C(═S)NH(C₁₋₆ alkyl), —C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₆alkyl, C₁₋₆perhaloalkyl, C₂₋₆alkenyl, C₂₋₆alkynyl, heteroC₁₋₆alkyl, heteroC₂₋₆alkenyl, heteroC₂₋₆alkynyl, C₃₋₁₀carbocyclyl, C₆₋₁₀aryl, 3-10 membered heterocyclyl, 5-10 membered heteroaryl; or two geminal R⁹⁹ substituents can be joined to form ═O or ═S; wherein X⁻ is a counterion.

The term “halo” or “halogen” refers to fluorine (fluoro, —F), chlorine (chloro, —Cl), bromine (bromo, —Br), or iodine (iodo, —I).

The term “acyl” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, —C(═S)O(R^{X1}), —C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or dialkylamino, mono- or di-heteroalkylamino, mono- or di-arylamino, or mono- or diheteroarylamino; or two R^{X1} groups taken together form a 5- to 6-membered heterocyclic ring.

Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “carbonyl” refers a group wherein the carbon directly attached to the parent molecule is sp² hybridized, and is substituted with an oxygen, nitrogen or sulfur atom, e.g., a group selected from ketones (e.g., —C(═O)R^{aa}), carboxylic acids (e.g., —CO₂H), aldehydes(CHO), esters (e.g., —CO₂R^{aa}, —C(═O)SR^{aa}, —C(═S)SR^{aa}), amides (e.g., —C(═O)N(R^{bb})₂, C(═O)NR^{bb}SO₂R^{aa}, —C(═S)N(R^{bb})₂, and imines (e.g., —C(═NR^{bb})R^{aa}, —C(═NR^{bb})OR^{aa}), C(═NR^{bb})N(R^{bb})₂, wherein R^{aa} and R^{bb} are as defined herein.

The term “oxo” refers to the group ═O, and the term “thiooxo” refers to the group ═S.

The term “cyano” refers to the group —CN.

The term “azide” and “azido” refers to the group —N₃.

Nitrogen atoms can be substituted or unsubstituted as valency permits, and include primary, secondary, tertiary, and quaternary nitrogen atoms. Exemplary nitrogen atom substituents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or a 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc}, and R^{dd} are as defined herein.

As used herein, the designation of a polyvalent moiety without specifying the specific order of attachment is intended to cover all possible arrangements. By way of example, a compound represented by the formula:

A-X-B,
wherein X is NHC(═O) embraces both:

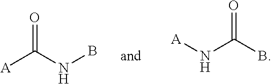

As used herein, a chemical bond depicted [symbol] represents either a single, double, or triple bond, valency permitting. By way of example,

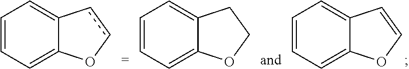

An electron-withdrawing group is a functional group or atom that pulls electron density towards itself, away from other portions of the molecule, e.g., through resonance and/or inductive effects. Exemplary electron-withdrawing groups include F, Cl, Br, I, NO₂, CN, SO₂R, SO₃R, SO₂NR₂, C(O)R^{1a}, C(O)OR, and C(O)NR₂ (wherein R is H or an alkyl, aryl, heteroaryl, cycloalkyl, heterocyclyl group) as well as alkyl group substituted with one or more of those group.

An electron-donating group is a functional group or atom that pushes electron density away from itself, towards other portions of the molecule, e.g., through resonance and/or inductive effects. Exemplary electron-donating groups include unsubstituted alkyl or aryl groups, OR and N(R)₂ and alkyl groups substituted with one or more OR and N(R)₂ groups.

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture. Unless stated to the contrary, a formula depicting one or more stereochemical features does not exclude the presence of other isomers.

Some compounds disclosed herein may exist as one or more tautomers. Tautomers are interconvertible structural isomers that differ in the position of one or more protons or other labile atom. By way of example:

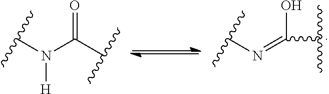

The prevalence of one tautomeric form over another will depend on the specific chemical compound as well as its local chemical environment. Unless specified to the contrary, the depiction of one tautomeric form is inclusive of all possible tautomeric forms.

Unless stated to the contrary, a substituent drawn without explicitly specifying the point of attachment indicates that the substituent may be attached at any possible atom. For example, in a benzofuran depicted as:

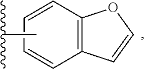

the substituent may be present at any one of the six possible carbon atoms.

As used herein, the term “null,” when referring to a possible identity of a chemical moiety, indicates that the group is absent, and the two adjacent groups are directly bonded to one another. By way of example, for a genus of compounds having the formula CH₃—X—CH₃, if X is null, then the resulting compound has the formula CH₃—CH₃.

### Compounds

Described herein are compounds of Formula I, IIa, IIb, IIc, and IId.

In some embodiments, described herein are compounds of Formula I

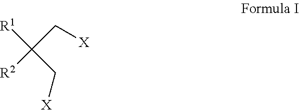

wherein:
X is a halogen; and
R¹ and R² are each independently selected from hydrogen, substituted or unsubstituted alkyl, cycloalkyl, alkoxy, aryl or heteroaryl, alkylaryl, ether, or R¹ and R², taken together with the atom to which they are directly attached form a four or more membered ring; and at least one of R¹ or R² are substituted or unsubstituted alkyl, cycloalkyl, alkoxy, aryl or heteroaryl, alkylaryl, or ether.

In some embodiments, described herein are compounds of Formula IIa

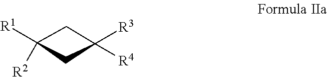

wherein:
R¹ and R² are each independently selected from hydrogen, substituted or unsubstituted alkyl, cycloalkyl, alkoxy, aryl or heteroaryl, alkylaryl, ether, or R¹ and R², taken together with the atom to which they are directly attached form a four or more membered ring; and at least one of R¹ or R² are substituted or unsubstituted alkyl, cycloalkyl, alkoxy, aryl or heteroaryl, alkylaryl, or ether; and R³ and R⁴ are ester.

In some embodiments, R³ and R⁴ are —CO₂Me.

In some embodiments, described herein are compounds of Formula IIb

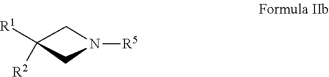

wherein:
R¹ and R² are each independently selected from hydrogen, substituted or unsubstituted alkyl, cycloalkyl, alkoxy, aryl or heteroaryl, alkylaryl, ether, or R¹ and R², taken together with the atom to which they are directly attached form a four or more membered ring; and at least one of R¹ or R² are substituted or unsubstituted alkyl, cycloalkyl, alkoxy, aryl or heteroaryl, alkylaryl, or ether; and
R⁵ is substituted or unsubstituted alkenyl, alkylaryl, aryl, or heteroaryl. In some embodiments, R⁵ is substituted or unsubstituted alkylaryl. In some embodiments, R⁵ is substituted or unsubstituted alkenyl. In some embodiments, R⁵ is benzyl.

In some embodiments, described herein are compounds of Formula IIc

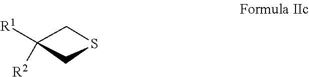

wherein:
R¹ and R² are each independently selected from hydrogen, substituted or unsubstituted alkyl, cycloalkyl, alkoxy, aryl or heteroaryl, alkylaryl, ether, or R¹ and R², taken together with the atom to which they are directly attached form a four or more membered ring; and at least one of R¹ or R² are substituted or unsubstituted alkyl, cycloalkyl, alkoxy, aryl or heteroaryl, alkylaryl, or ether.

In some embodiments, described herein are compounds of Formula IId

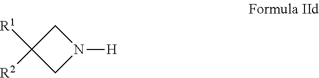

wherein:
R¹ and R² are each independently selected from hydrogen, substituted or unsubstituted alkyl, cycloalkyl, alkoxy, aryl or heteroaryl, alkylaryl, ether, or R¹ and R², taken together with the atom to which they are directly attached form a four or more membered ring; and at least one of R¹ or R² are substituted or unsubstituted alkyl, cycloalkyl, alkoxy, aryl or heteroaryl, alkylaryl, or ether.

In some embodiments, R¹ is substituted or unsubstituted alkylaryl. In some embodiments, R² is substituted or unsubstituted hydrogen, alkyl, cycloalkyl, or alkoxy. In some embodiments, R¹ and R², taken together with the atom to which they are directly attached form a four or more membered ring.

In some embodiments, R¹ and R², taken together with the atom to which they are directly attached form the ring

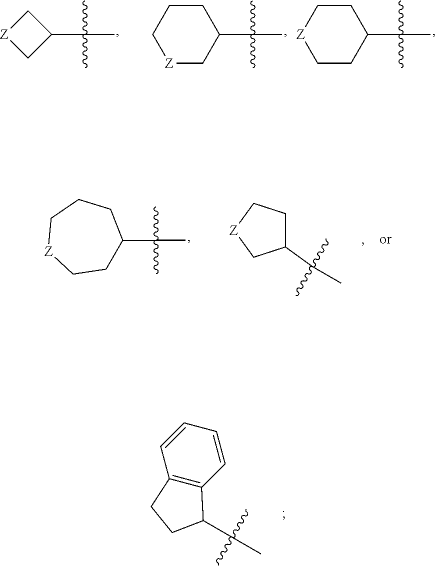

wherein
Z is O, CR⁶R⁷, or NR⁸;
R⁶ and R⁷ are each independently H, or halogen; and R⁸ is a protecting group.

In some embodiments, the protecting group is tert-butyloxycarbonyl group (Boc).

In some embodiments, R¹ and R², taken together with the atom to which they are directly attached form the ring

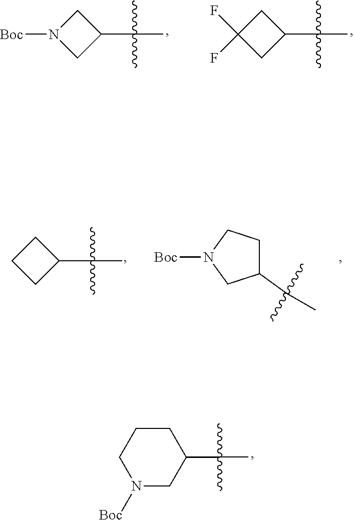

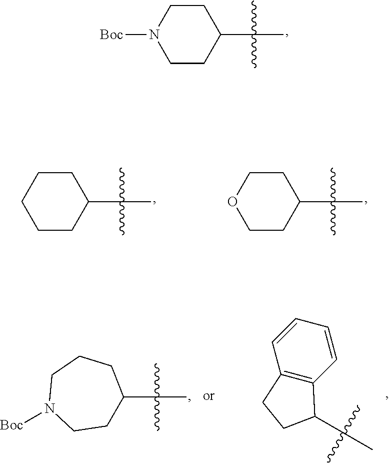

In some embodiments, the compound of Formula I is selected from:

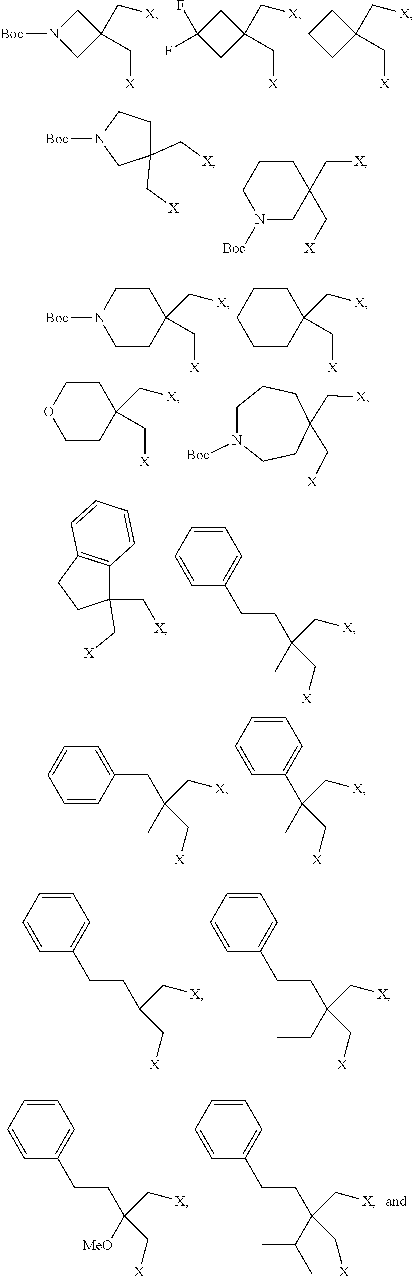

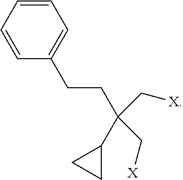

In some embodiments, compounds of Formula IIa, IIb, IIc, or IId can be selected from:

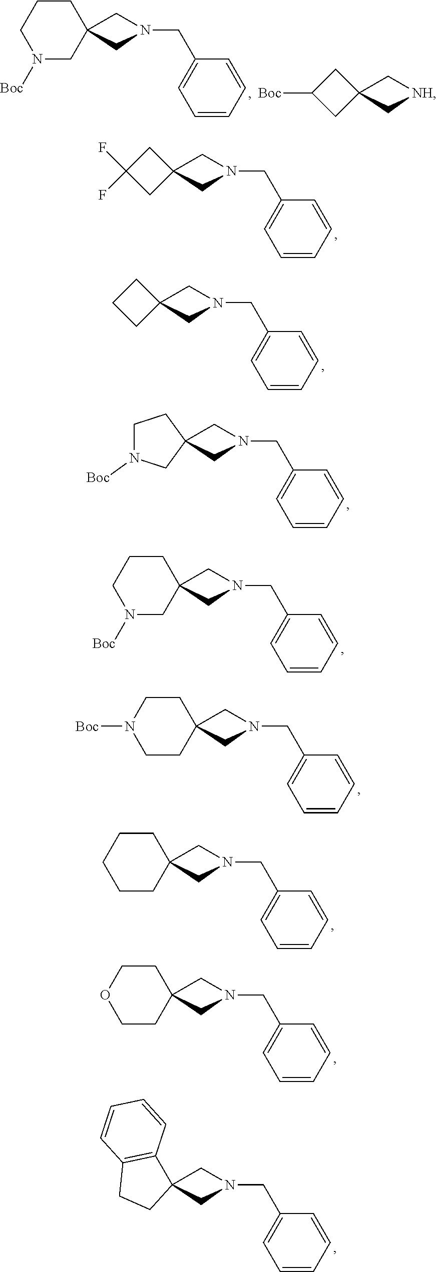

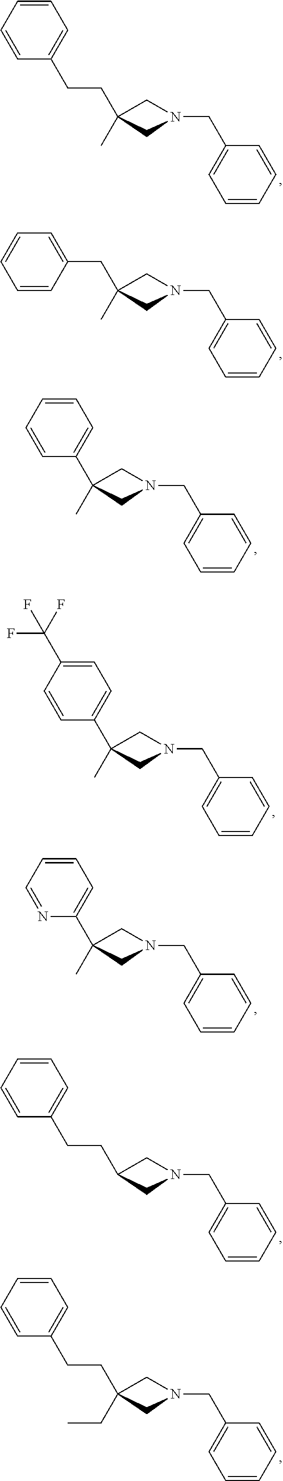

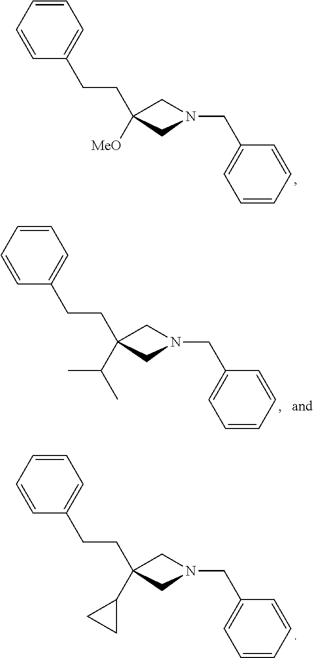

### Methods

Provided herein are processes and intermediates useful for the preparation of compounds of Formula I, IIa, IIb, IIc, and IId.

In some embodiments, described herein is a process for preparing a compound of Formula I

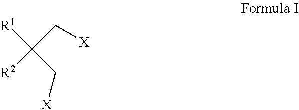

comprising:
(a) treating a compound of formula 10

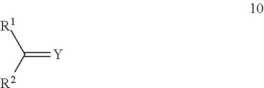

with a compound of formula 11

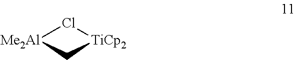

to form a first intermediate and treating the first intermediate with M₂ to form the compound of Formula I;
wherein:
X is a halogen;
Y is O or CH₂;
M is Cl, Br, or I; and
R¹ and R² are each independently selected from hydrogen, substituted or unsubstituted alkyl, cycloalkyl, alkoxy, aryl or heteroaryl, alkylaryl, ether, or R¹ and R², taken together with the atom to which they are directly attached form a four or more membered ring; and at least one of R¹ or R² are substituted or unsubstituted alkyl, cycloalkyl, alkoxy, aryl or heteroaryl, alkylaryl, or ether.

In some embodiments, the process further includes isolating the compound of Formula I.

In some embodiments, the process can include a solvent. In some embodiments, the solvent can be an aprotic solvent. Suitable aprotic solvents can include, but are not limited to, acetone, DMSO, DMF, THF, dichloromethane, acetonitrile, or any combination thereof. In some embodiments, the treating step is performed at 0° C.

In some embodiments, the first intermediate is defined by the structure below:

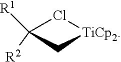

In some embodiments, the compound of formula 10 is selected from:

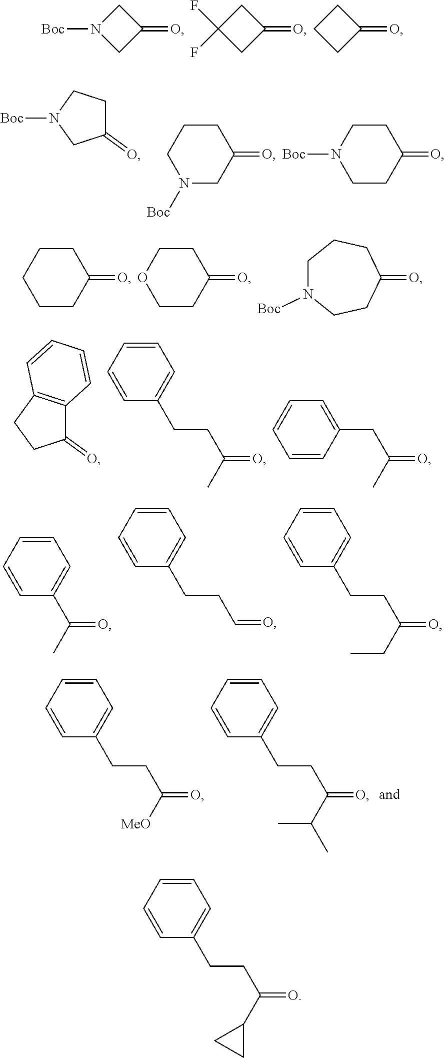

In some embodiments, Y is O. In some embodiments, Y is CH₂. In some embodiments, R¹ is substituted or unsubstituted aryl. In some embodiments, R¹ is substituted or unsubstituted alkylaryl. In some embodiments, R² is substituted or unsubstituted hydrogen, alkyl, cycloalkyl, or alkoxy. In some embodiments, R¹ and R², taken together with the atom to which they are directly attached form a four or more membered ring.

In some embodiments, R¹ and R², taken together with the atom to which they are directly attached form the ring

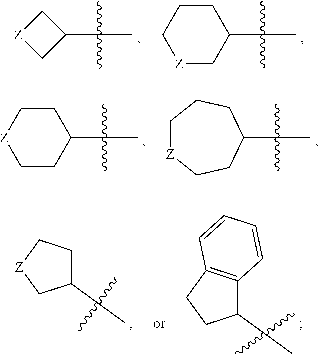

wherein
Z is O, CR⁶R⁷, or NR⁸;
R⁶ and R⁷ are each independently H, or halogen; and
R⁸ is a protecting group.

In some embodiments, the protecting group is tert-butyloxycarbonyl group (Boc).

In some embodiments, R¹ and R², taken together with the atom to which they are directly attached form the ring

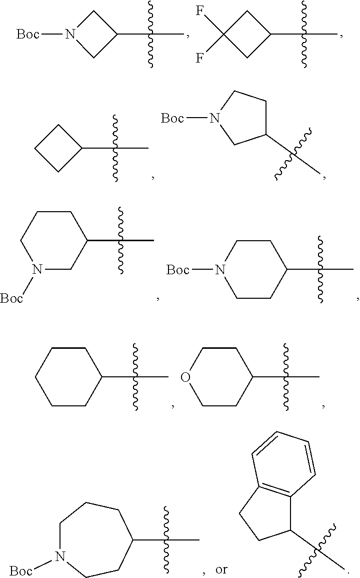

In some embodiments, the compound of Formula I is selected from:

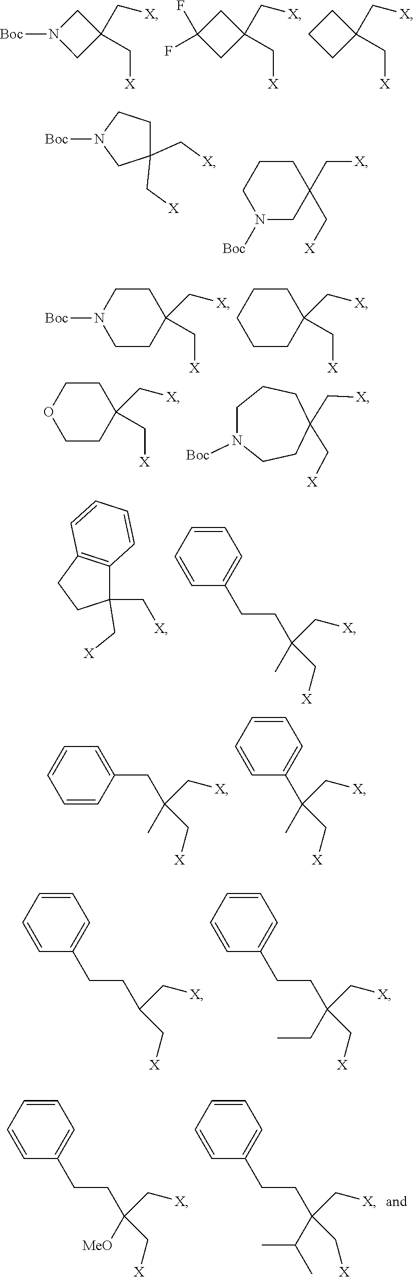

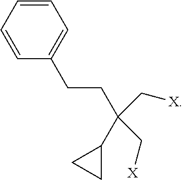

In some embodiments, described herein is a process for preparing a compound of Formula IIa

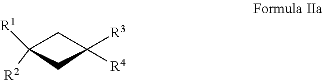

comprising:
(a) treating a compound of Formula I

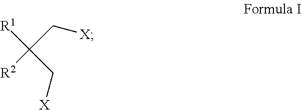

with

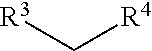

in the presence of a base to form a compound of Formula IIa;
wherein:
X is a halogen;
R¹ and R² are each independently selected from hydrogen, substituted or unsubstituted alkyl, cycloalkyl, alkoxy, aryl or heteroaryl, alkylaryl, ether, or R¹ and R², taken together with the atom to which they are directly attached form a four or more membered ring; and at least one of R¹ or R² are substituted or unsubstituted alkyl, cycloalkyl, alkoxy, aryl or heteroaryl, alkylaryl, or ether; and
R³ and R⁴ are ester.

In some embodiments, the process for preparing a compound of Formula IIa can include preparing a compound of Formula I as described herein; and treating the compound of Formula I with

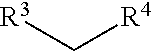

in the presence of a base to form a compound of Formula IIa. In some embodiments, R³ and R⁴ are —CO₂Me. In some embodiments,

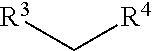

can be

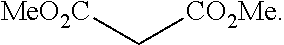

In some embodiments, the process can further include isolating the compound of Formula IIa.

In some embodiments, the process can be performed in a solvent. In some embodiments, the solvent can be an aprotic solvent. Suitable aprotic solvents can include, but are not limited to, acetone, DMSO, DMF, THF, dichloromethane, acetonitrile, or any combination thereof. In some embodiments, the treating step is performed at 80° C. In some embodiments, the treating step further comprises an iodide ion. In some embodiments, the base is K₂CO₃.

In some embodiments, R¹ is substituted or unsubstituted alkylaryl. In some embodiments, R² is substituted or unsubstituted hydrogen, alkyl, cycloalkyl, or alkoxy. In some embodiments, R¹ and R², taken together with the atom to which they are directly attached form a four or more membered ring.

In some embodiments, R¹ and R², taken together with the atom to which they are directly attached form the ring

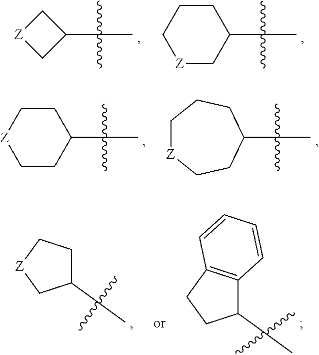

wherein
Z is O, CR⁶R⁷, or NR⁸;
R⁶ and R⁷ are each independently H, or halogen; and
R⁸ is a protecting group.

In some embodiments, the protecting group is tert-butyloxycarbonyl group (Boc).

In some embodiments, R¹ and R², taken together with the atom to which they are directly attached form the ring

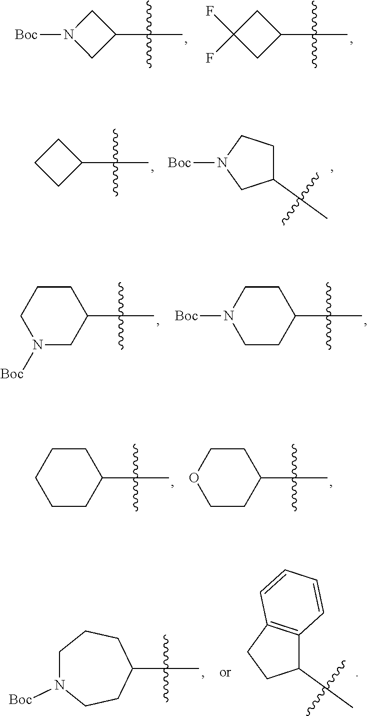

In some embodiments, described herein is also a process for preparing a compound of Formula IIb

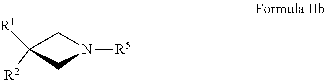

comprising:
(a) treating a compound of Formula I

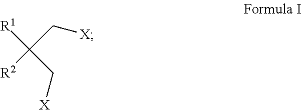

with R⁵—NH₂ in the presence of a base to form a compound of Formula IIb;
wherein:
X is a halogen;
R¹ and R² are each independently selected from hydrogen, substituted or unsubstituted alkyl, cycloalkyl, alkoxy, aryl or heteroaryl, alkylaryl, ether, or R¹ and R², taken together with the atom to which they are directly attached form a four or more membered ring; and at least one of R¹ or R² are substituted or unsubstituted alkyl, cycloalkyl, alkoxy, aryl or heteroaryl, alkylaryl, or ether; and
R⁵ is substituted or unsubstituted alkenyl, alkylaryl, aryl, or heteroaryl.

In some embodiments, R⁵ is substituted or unsubstituted alkylaryl. In some embodiments, R⁵ is substituted or unsubstituted alkenyl. In some embodiments, R⁵ is benzyl.

In some embodiments, the process for preparing a compound of Formula IIb can include preparing a compound of Formula I as described herein; and treating the compound of Formula I with R⁵—NH₂ in the presence of a base to form a compound of Formula IIb.

In some embodiments, the process can further include isolating the compound of Formula IIb.

In some embodiments, the process can be performed in a solvent. In some embodiments, the solvent can be an aprotic solvent. Suitable aprotic solvents can include, but are not limited to, acetone, DMSO, DMF, THF, dichloromethane, acetonitrile, or any combination thereof. In some embodiments, the treating step is performed at 80° C. In some embodiments, the treating step further comprises an iodide ion. In some embodiments, the base is K₂CO₃.

In some embodiments, R¹ is substituted or unsubstituted alkylaryl. In some embodiments, R² is substituted or unsubstituted hydrogen, alkyl, cycloalkyl, or alkoxy. In some embodiments, R¹ and R², taken together with the atom to which they are directly attached form a four or more membered ring.

In some embodiments, R¹ and R², taken together with the atom to which they are directly attached form the ring

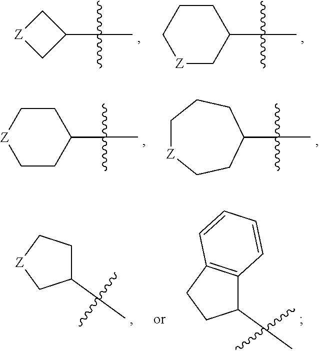

wherein
Z is O, CR⁶R⁷, or NR⁸;
R⁶ and R⁷ are each independently H, or halogen; and
R⁸ is a protecting group.

In some embodiments, the protecting group is tert-butyloxycarbonyl group (Boc).

In some embodiments, R¹ and R², taken together with the atom to which they are directly attached form the ring

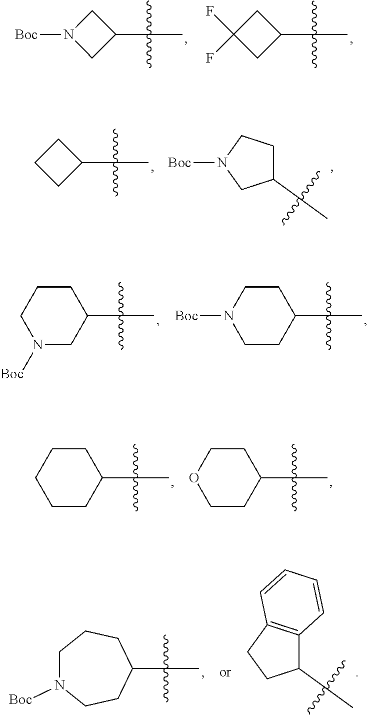

In some embodiments, R⁵ is substituted or unsubstituted alkylaryl. In some embodiments, R⁵ is substituted or unsubstituted alkenyl. In some embodiments, R⁵ is benzyl.

In some embodiments, described herein is also a process for preparing a compound of Formula IIc

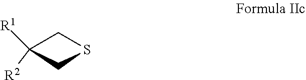

comprising:
(b) treating a compound of Formula I

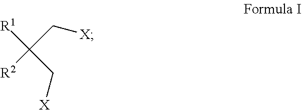

with Na₂S to form a compound of Formula IIc;
wherein:
X is a halogen;
R¹ and R² are each independently selected from hydrogen, substituted or unsubstituted alkyl, cycloalkyl, alkoxy, aryl or heteroaryl, alkylaryl, ether, or R¹ and R², taken together with the atom to which they are directly attached form a four or more membered ring; and at least one of R¹ or R² are substituted or unsubstituted alkyl, cycloalkyl, alkoxy, aryl or heteroaryl, alkylaryl, or ether.

In some embodiments, the process for preparing a compound of Formula IIc can include preparing a compound of Formula I as described herein; and treating the compound of Formula I with Na₂S to form a compound of Formula IIc.

In some embodiments, the process can further include isolating the compound of Formula IIc.

In some embodiments, the process can be performed in a solvent. In some embodiments, the solvent can be an aprotic solvent. Suitable aprotic solvents can include, but are not limited to, acetone, DMSO, DMF, THF, dichloromethane, acetonitrile, or any combination thereof. In some embodiments, the treating step is performed at 50° C.

In some embodiments, R¹ is substituted or unsubstituted alkylaryl. In some embodiments, R² is substituted or unsubstituted hydrogen, alkyl, cycloalkyl, or alkoxy. In some embodiments, R¹ and R², taken together with the atom to which they are directly attached form a four or more membered ring.

In some embodiments, R¹ and R², taken together with the atom to which they are directly attached form the ring

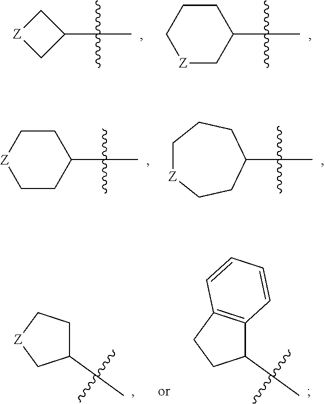

wherein
Z is O, CR⁶R⁷, or NR⁸;
R⁶ and R⁷ are each independently H, or halogen; and
R⁸ is a protecting group.

In some embodiments, the protecting group is tert-butyloxycarbonyl group (Boc).

In some embodiments, R¹ and R², taken together with the atom to which they are directly attached form the ring

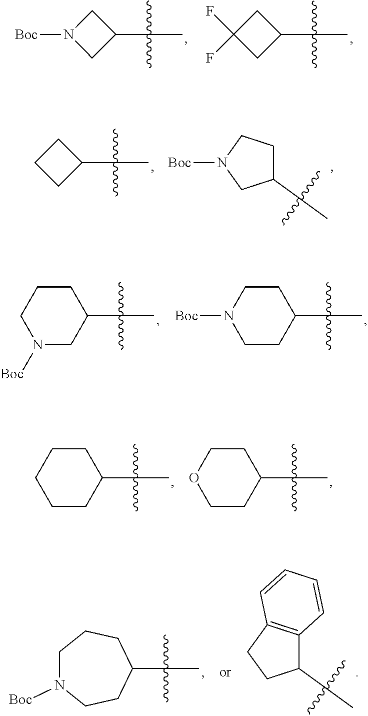

In some embodiments, described herein is a process for preparing a compound of Formula IId

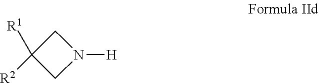

comprising:
(a) treating a compound of Formula I

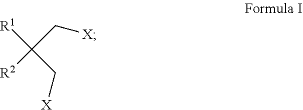

with R⁵—NH₂ in the presence of a base to form a compound of Formula IIb; and
(b) hydrogenolysis of the compound of Formula IIb to form a compound of Formula IId;
wherein:
X is a halogen;
R¹ and R² are each independently selected from hydrogen, substituted or unsubstituted alkyl, cycloalkyl, alkoxy, aryl or heteroaryl, alkylaryl, ether, or R¹ and R², taken together with the atom to which they are directly attached form a four or more membered ring; and at least one of R¹ or R² are substituted or unsubstituted alkyl, cycloalkyl, alkoxy, aryl or heteroaryl, alkylaryl, or ether; and
R⁵ is substituted or unsubstituted alkenyl, alkylaryl, aryl, or heteroaryl.

In some embodiments, R⁵ is substituted or unsubstituted alkylaryl. In some embodiments, R⁵ is substituted or unsubstituted alkenyl. In some embodiments, R⁵ is benzyl.

In some embodiments, the process for preparing a compound of Formula IId can include:
a) preparing a compound of Formula I as described herein;
b) treating the compound of Formula I with R⁵—NH₂ in the presence of a base to form a compound of Formula IIb; and
c) hydrogenolysis of the compound of Formula IIb to form a compound of Formula IId;
wherein
R⁵ is substituted or unsubstituted alkenyl, alkylaryl, aryl, or heteroaryl.

In some embodiments, R⁵ is substituted or unsubstituted alkylaryl. In some embodiments, R⁵ is substituted or unsubstituted alkenyl. In some embodiments, R⁵ is benzyl.

In some embodiments, the process for preparing a compound of Formula IId can include:
a) preparing a compound of Formula IIb as described herein; and
b) hydrogenolysis of the compound of Formula IIb to form a compound of Formula IId.

In some embodiments, the process can further include isolating the compound of Formula IId.

In some embodiments, the process can be performed in a solvent. In some embodiments, the solvent can be an aprotic solvent. Suitable aprotic solvents can include, but are not limited to, acetone, DMSO, DMF, THF, dichloromethane, acetonitrile, or any combination thereof. In some embodiments, the treating step is performed at 50° C.

In some embodiments, R¹ is substituted or unsubstituted alkylaryl. In some embodiments, R² is substituted or unsubstituted hydrogen, alkyl, cycloalkyl, or alkoxy. In some embodiments, R¹ and R², taken together with the atom to which they are directly attached form a four or more membered ring.

In some embodiments, R¹ and R², taken together with the atom to which they are directly attached form the ring

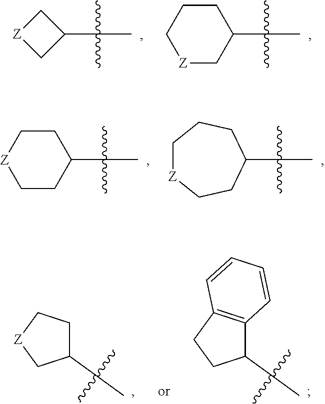

wherein
Z is O, CR⁶R⁷, or NR⁸;
R⁶ and R⁷ are each independently H, or halogen; and
R⁸ is a protecting group.

In some embodiments, the protecting group is tert-butyloxycarbonyl group (Boc).

In some embodiments, R¹ and R², taken together with the atom to which they are directly attached form the ring

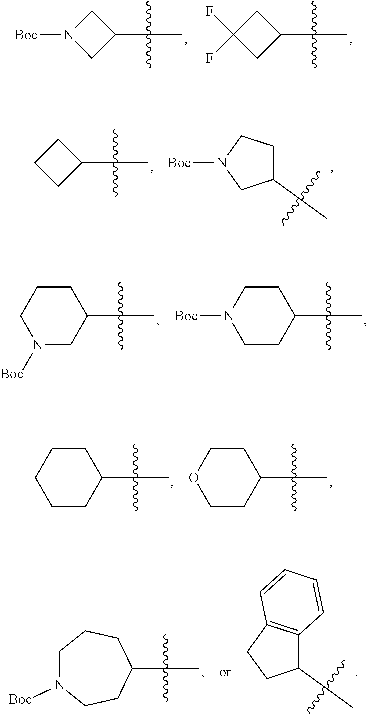

In some embodiments, the compound of Formula I is selected from:

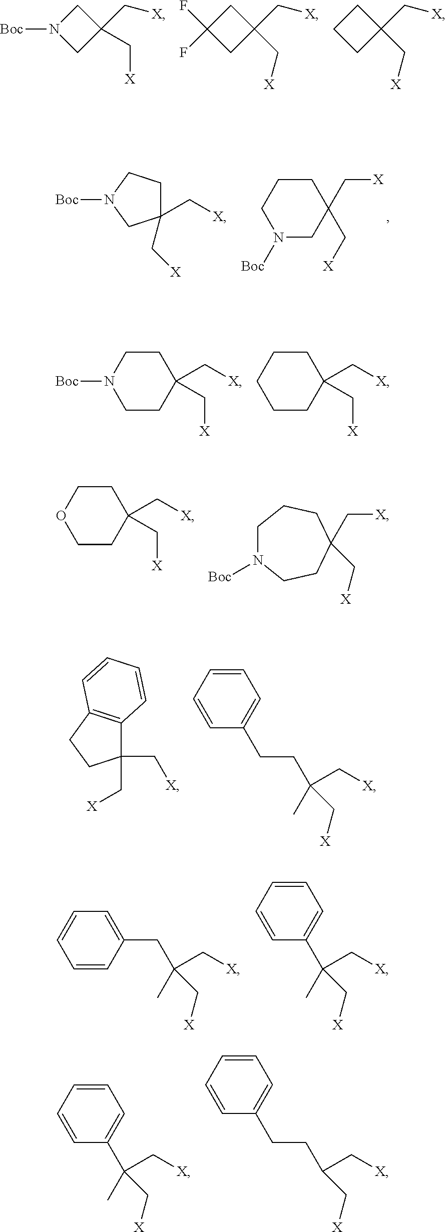

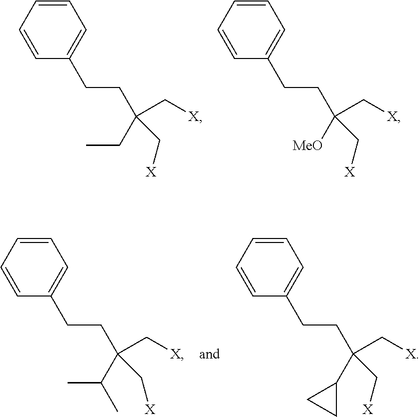

In some embodiments, the compound of Formula IIa, IIb, IIc, or IId can be selected from:

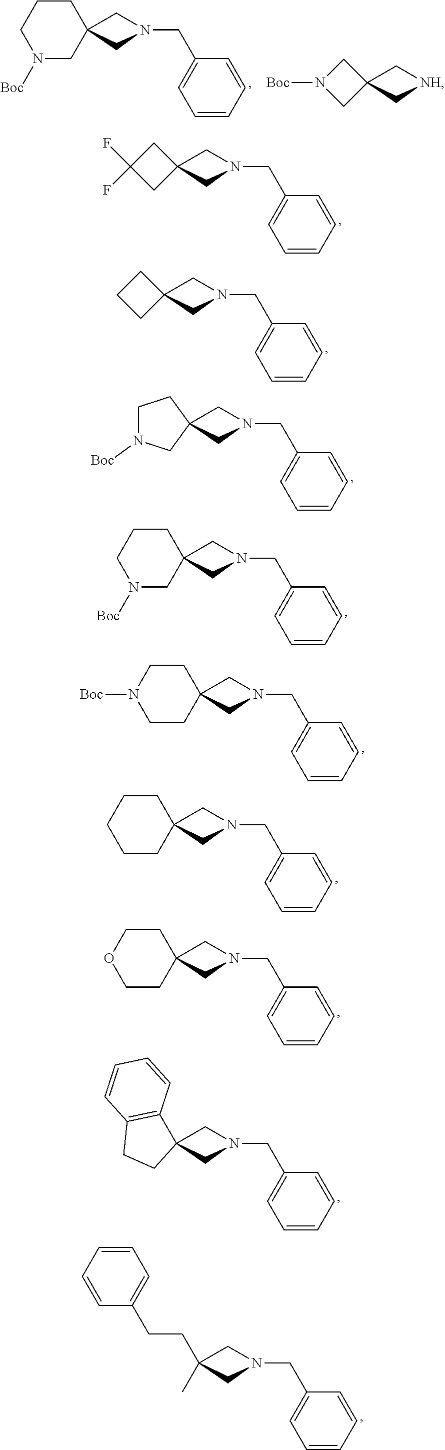

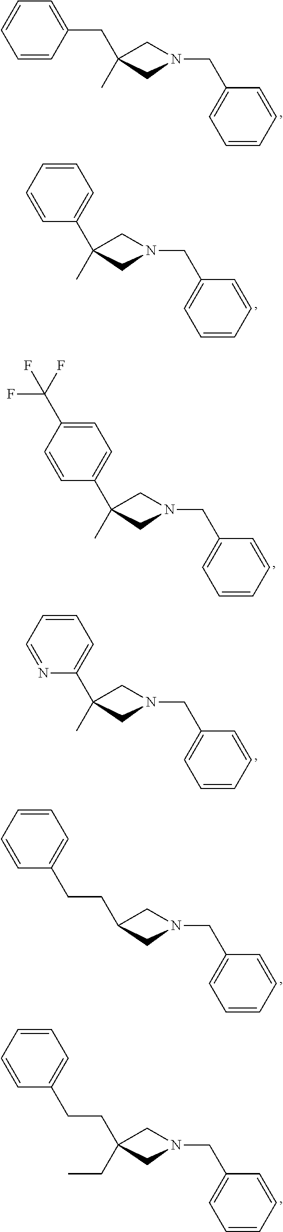

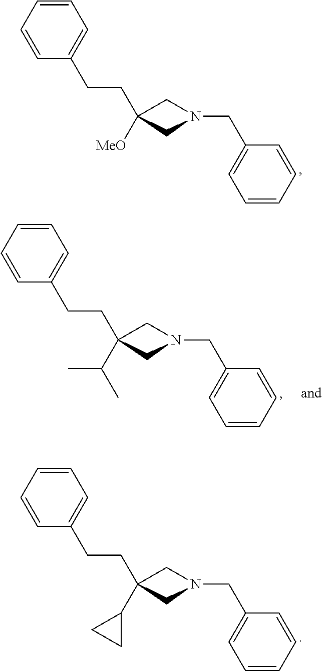

Variations on compounds used in the processes for the preparation of compounds of Formula I can include the addition, subtraction, or movement of various constituents as described for each compounds. Similarly, when one or more chiral centers is present in a molecule, the chirality of the molecule can be changed. Additionally, the synthesis of the compounds used in these processes can involve the protection of various chemical groups, and further the compounds prepared by the disclosed processes may be subsequently deprotected as appropriate. The use of protection and deprotection, and the selection of appropriate protecting groups, would be readily known to one skilled in the art. “Protecting group”, as used herein, refers to any convention functional group that allows one to obtain chemoselectivity in a subsequent chemical reaction. Protecting groups are described, for example, in Peter G. M. Wuts, Greene's Protective Groups in Organic Synthesis, 5th Ed., Wiley & Sons, 2014. For a particular compound and/or a particular chemical reaction, a person skilled in the art knows how to select and implement appropriate protecting groups and their associated synthetic methods. Examples of amine protecting groups include acyl and alkoxy carbonyl groups, such a t-butoxycarbonyl (BOC) and [2-(trimethylsilyl)ethoxy]methoxy (SEM). Examples of carboxyl protecting groups include C1-C6 alkoxy groups, such as methyl, ethyl, and t-butyl. Examples of alcohol protecting groups include benzyl, trityl, silyl ethers, and the like.

The described processes, or reaction to produce the compounds used in the described processes, can be carried out in solvents indicated herein, or in solvents which can be selected by one of skill in the art of organic synthesis. Solvents can be substantially nonreactive with the starting materials (reactants), intermediates, or products under the conditions at which the reaction is carried out, i.e., temperature and pressure. Reactions can be carried out in one solvent or a mixture of more than one solvent. Product or intermediate formation can be monitored according to any suitable method known in the art. For example, product formation can be monitored by spectroscopic means, such as nuclear magnetic resonance spectroscopy (e.g., 1H and 13C), infrared spectroscopy, spectrophotometry (e.g., UV-visible), or mass spectrometry, or by chromatography such as high performance liquid chromatography (HPLC) or thin layer chromatography (TLC).

### EXAMPLES

The following examples are for the purpose of illustration of the invention only and are not intended to limit the scope of the present invention in any manner whatsoever.

### Example 1: Constructing all-Carbon Quaternary Centers from Ketones Via Titanacyclobutanes: Rapid Access to Azaspiro[3.n]Alkanes

Quaternary carbon centers pose a significant challenge in chemical synthesis. Harnessing the underexplored reactivity of titanacyclobutane intermediates a strategy to construct functionalized all-carbon quaternary centers from ketones is described. This method streamlines access to azaspiro[3.n]alkanes and 3-azetidines, a class of four membered ring-containing heterocycles that have emerged as valuable three-dimensional inputs for drug discovery.

Access to the linchpin quaternary carbon center of azaspiro[3.n]alkanes using Cp₂Ti(μ-Cl)(μ-CH₂)AlMe₂ (3; Cp=C₅H₅) as a progenitor to titanacyclobutanes 4 was envisioned (Scheme 1) (Tebbe, F. N., et al., J. Am. Chem. Soc. 1978, 100, 3611). Building upon pioneering studies from Tebbe (Tebbe, F. N., et al., J. Am. Chem. Soc. 1979, 101, 5074) and Grubbs (Howard, T. R., et al., J. Am. Chem. Soc. 1980, 102, 6876; Lee, J. B., et al., J. Am. Chem. Soc. 1982, 104, 7491), it was previously demonstrated that the degenerate metathesis equilibria between the titanocene methylidene unveiled from 3 (i.e., Cp₂TiCH₂) and C—C π-bonds could be used for alkene hydromethylation (Law, J. A., et al., Angew. Chem., Int. Ed. 2021, 60, 14360 (“Law, et al., 2021”)). This methodology exploited 4 as a 1,3-dianion equivalent. In the present context, it was reasoned that 4 could be generated directly from ketones via sequential carbonyl methylenation and alkene cyclometallation if an excess of 3 was employed (Hanzawa, Y., et al., Tetrahedron 1998, 54, 11387). Halogenation of 4 might then produce dihalide 5 (Brown-Wensley, K. A., et al., Pure Appl. Chem. 1983, 55, 1733), which could be reacted subsequently with an amine to furnish 3-azetidines. The potential of this sequence to rapidly construct azaspiro[3.n]alkanes from readily available cyclic ketones was apparent. Moreover, the need for excess quantities of 3 was justified by direct access to quaternary carbon fragments that are difficult to prepare. The estimated cost to prepare Tebbe's reagent (3) is ˜$1.3/mmol starting from Cp₂TiCl₂ and AlMe₃. Details for the preparation of 3 used in this study are outlined in reference Law, et al., 2021. Preparing 3 within 1-2 weeks of use is recommended (Law, et al., 2021).

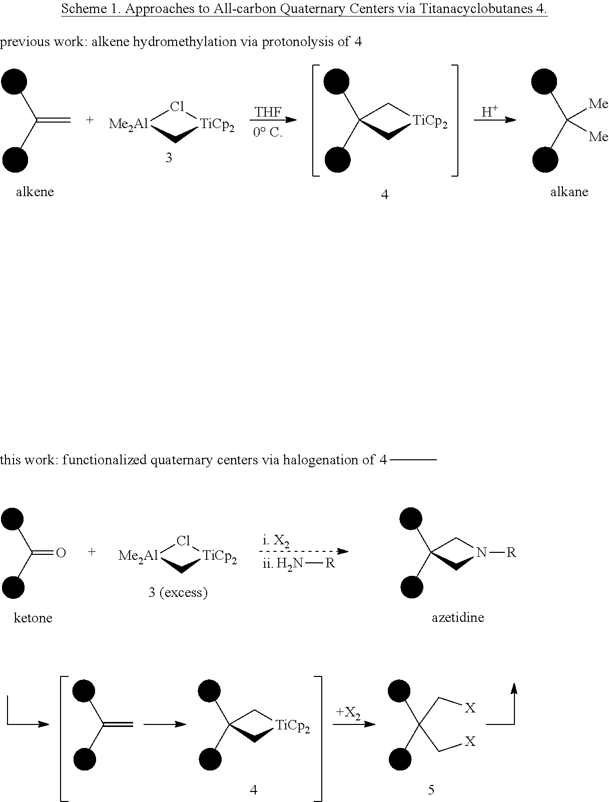

Commercially available N-Boc-3-azetidenone (6) was identified as an initial substrate to establish the feasibility of this idea. N-Boc-3-azetidinone (6) was purchased from Biosynth [FB18887] for $0.9/gram. Thus, extending our previous study (Law, et al., 2021), reacting 6 with 3 equiv of 3 in THF (0.3 M) at 0° C. resulted in quantitative formation of titanacyclobutane 4a after 1 h. Protonolysis of 4a prepared from 6 under these conditions afforded gem-dimethyl product 9 in 93% isolated yield (>95% conversion). Initial attempts to intercept 4a with various common halogen sources (e.g. NCS, NBS, etc.) gave complex mixtures of dihalide 8 alongside products 9-11 (Table 1). See Table S1 for a complete survey of electrophiles. The most useful electrophiles from this initial screen were I₂ and Br₂, which returned mixtures of 8 and cyclopropane 10 after workup. These results can be rationalized by addition of X₂ across a Ti—C bond to produce Ti(IV) complex 7 (Suzzy, C. H., et al., J. Am. Chem. Soc. 1984, 106, 1533). As a result, structures 8 and 11 arise from competing S_{N}2 processes involving (i) intermolecular capture of X₂ by 7 to give 8 and (ii) an intramolecular 3-exo-tet cyclization within 7 to afford 11. Consistent with this model, the ratio of 8:11 was dependent on the identity and stoichiometry of X₂. For example, whereas I₂ favored the formation of 11 over 8a (X=I, entries 1-3), Br₂ produced 8b (X=Br) as the major product. Assuming that the cyclization of 7 was slower when X=Br than when X=I, the stoichiometry of Br₂ was increased to improve the yield of 8b (entries 4-7). Accordingly, 10 equiv of Br₂ gave 8b exclusively in 87% isolated yield (>95% conversion, >20:1 ratio of 8:11).

TABLE 1
Optimizing the Halogenation of Titanacyclobutane 4a.

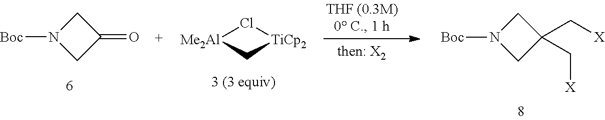

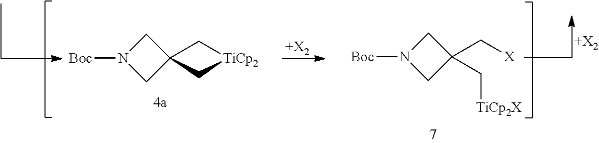

observed side products
9

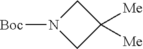 [SMILES: CC1(C)CN(C(=O)OC(C)(C)C)C1]

10

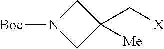

11

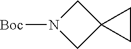 [SMILES: CC(C)(C)OC(=O)N1CC2(CC2)C1]

| entry | X₂ (equiv)^{a} | 8 | X | 8:11 ratio^{b} | conversion to 8 (%) |
|---|---|---|---|---|---|
| 1 | I₂ (2) | 8a | I | 0:1 | 0 |
| 2 | I₂ (3) | 8a | I | 1:15 | 6 |
| 3 | I₂ (5) | 8a | I | 1:4 | 17 |
| 4 | Br₂ (3) | 8a | Br | 2:1 | 45 |
| 5 | Br₂ (5) | 8b | Br | 3:1 | 68 |
| 6 | Br₂ (7) | 8b | Br | 7:1 | 85 |
| 7 | Br₂ (10) | 8b | Br | >20:1 | 95 (87)^{c} |

^{a}Solutions of X₂ in CH₂Cl₂ or THF (1.0M) were added via syringe to 4a cooled to-78° C. After 0.25 h, the reaction was warmed to 0° C. for 0.75 h before addition of SiO₂ in Et₂O.
^{b}The ratio of 8:11 was determined by ¹H NMR.
^{c}Isolated yield (% based on 6).

To further investigate the reactivity of titanacyclobutane 4a, the effectiveness of I₂ and benzyl iodide (BnI) as electrophiles (Scheme 2) was compared. As anticipated, cyclopropane 11 was generated in 75% yield by treating 4a with 3 equiv of I₂ at at 0° C. This outcome is consistent with the facile intramolecular cyclization of Ti(IV) complex 7a. Alternatively, when 4a was treated with BnI (5 equiv), selective formation of monoiodide 10a (X=I, 68% yield) was observed alongside a stoichiometric quantity of bibenzyl. This outcome suggested a radical pathway that likely intercepted alkyl Ti(IV) complex 12. Evidence for 12 was obtained by repeating the reaction with 2-iodomethyl naphthalene (13). Following a workup with DCl in D₂O, this modification allowed for the simplified detection of 1,2-bis-(2-naphthyl)ethane and deuterated 2-methylnaphthalene (d-14) in the reaction mixture by ¹H NMR and facilitated the isolation of d-10a in 74% yield. Only traces of 11 were formed under these conditions. Thus, in contrast to 7a, alkyl Ti(IV) complex 12 was persistent at 0° C. Taken together, these observations illustrate the redox reactivity of titanacyclobutanes, which might involve outer-shell electron transfer between 4a and BnI (Burk, M. J., et al., J. Am. Chem. Soc. 1990, 112, 6133; Buchwald, S. L., et al., J. Am. Chem. Soc. 1985, 107, 1766). It also establish a selective entry point to any of products 8-11 based on the reaction conditions employed.

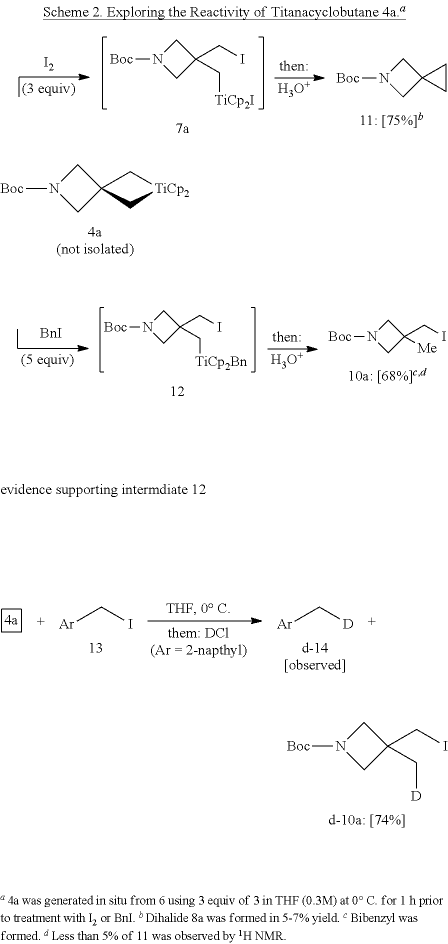

With direct access to 8b from ketone 6 in place, improving the assembly of azaspiro[3.3]heptane derivatives was a focus. First, short synthesis of 2,6-diazaspiro[3.3]heptane fragment 16 (Scheme 3) (Burkhard, J., et al., Org. Lett. 2008, 10, 3525 (“Burkhard, et al., 2008”)). Thus, 6 was converted to 4a as noted above, then reacted with Br₂ to give 8b in 84% yield on gram-scale. Cyclization of 8b with benzylamine afforded protected spirocycle 15 in 89% yield. Hydrogenolysis of 15 then provided 16 as a colorless solid in 87% yield. This material was stored for several weeks at 23° C. without incident; however, long-term storage resulted in slow, non-specific decomposition. This problem can be avoided by converting 16 to the corresponding oxalate salt, which was previously prepared in 8 steps (Burkhard, et al., 2008).

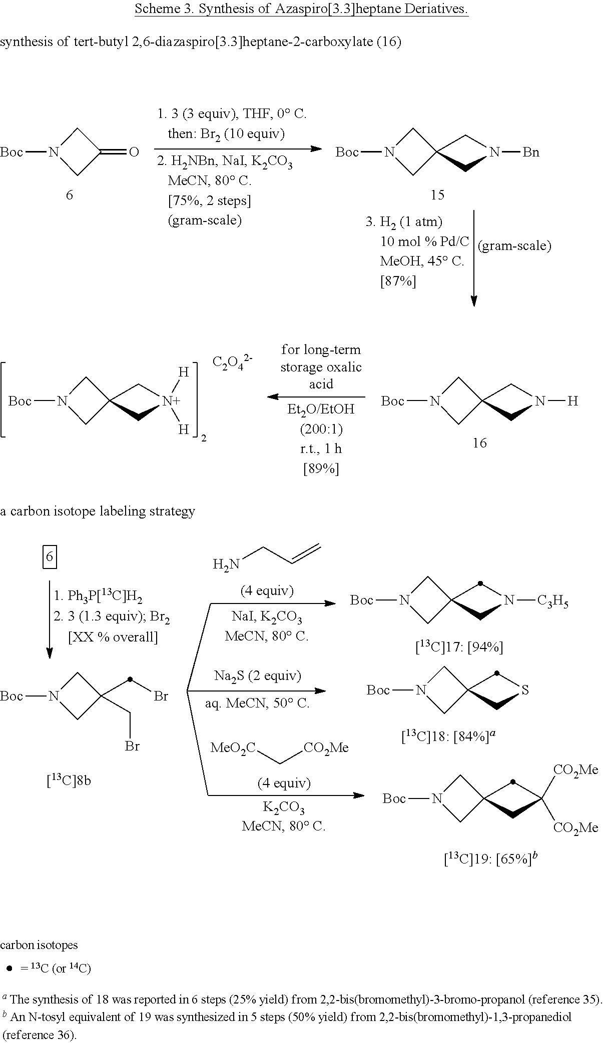

The availability of 8b in a single step also streamlines access to other in-demand azaspiro[3.3]heptanes. Existing approaches to this family avoid building the congested quaternary carbon center. For example, the reported 6-step synthesis of 8b starts from diethyl bis(hydroxymethyl)malonate (Miller, R. A., et al., Synth. Commun. 2003, 33, 3347; Wu, H.; European Patent Publication No. 3805217A1, Apr. 14, 2021). The N-tosyl congener of 8b was prepared in a similar manner in 4 steps (Bukhard, J. A.; Angew. Chem., Int. Ed. 2010, 49, 3524). In contrast, by constructing the quaternary carbon center directly our approach expedited the diversification of 6 into a series of azasprio[3.3]heptanes (i.e., 17-19, 2 steps each from 6) that are laborious to prepare using known chemistry (Borst, M. L., et al., ACS Comb. Sci. 2018, 20, 335; Radchenko, D. S., et al., J. Org. Chem. 2010, 75, 5941). As highlighted in Scheme 3, the carbon-13 isotopologs of 17-19 were also easily accessible by initially reacting ketone 6 with ¹³C-enriched methylenetriphenylphosphorane. The resultant alkene was then elaborated to [¹³C]8b in XX % yield using 1.3 equiv of 3. This intermediate provided isotopically labeled spirocycles that are inaccessible any other way. In principle, this approach can also be used to install a ¹⁴C-radiolabel to facilitate metabolism and disposition studies (Rhee, S.-W., et al., J. Label. Compd. Radiopharm. 2012, 55, 186; Babin, V., et al., JACS Au 2022, 2, 1234).

To complete a modular entry point to azaspiro[3.n]alkanes, the scope of alternative cyclic ketones (Scheme 4) was explored. Leveraging a slight modification of the 2-step procedure that minimized purification of the initial dihalide, N-Boc-3-pyrrolidinone and -piperidone afforded the orthogonally protected 2,6-diazaspiro[3.n]alkane scaffolds 20 and 21 in 64% and 63% overall yield (2-steps), respectively. Alternatively, N-Boc-4-azacycloheptanone furnished dihalide 22 in 64% yield; however, this species could not be cyclized, likely because the resultant heterocycle is too strained. Conversely, azaspirocycles 23-25 derived from various 6-membered (hetero)cyclic ketones were prepared in good yield. This approach was also compatible with cyclobutanone and 2,2-difluorocyclobutanone, which gave 2-azaspiro[3.3]heptane derivatives 26 and 27 in 58% and 57% yield, respectively. Surprisingly, aromatic cyclic ketones were more problematic. For example, 1-indanone was not consumed in the reaction, despite affording azetidine 28 in 34% yield. This result indicated a sluggish carbonyl methylenation step between Cp₂TiCH₂ and aromatic ketones.

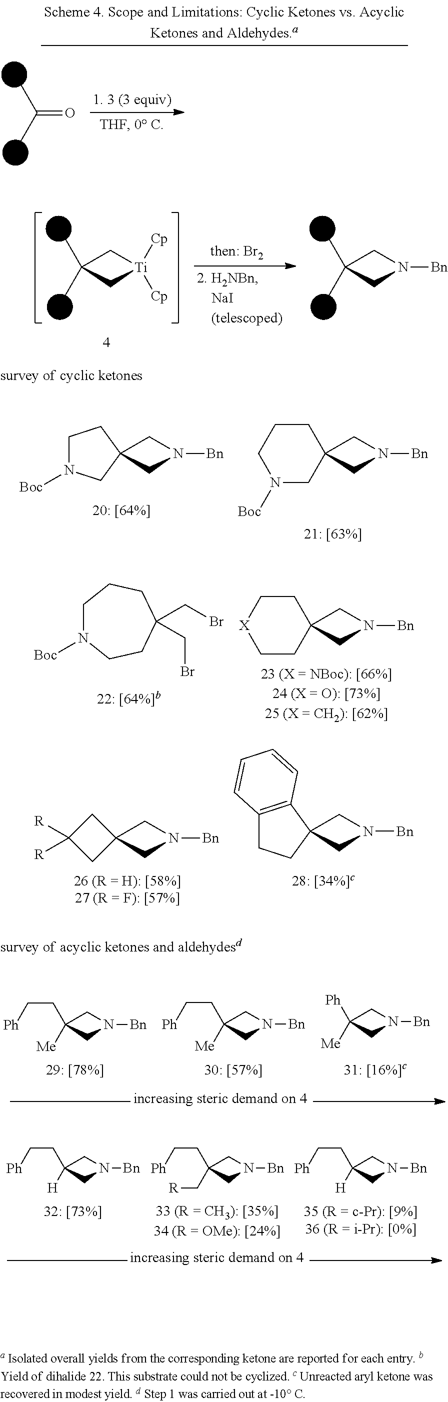

Notably, whereas cyclic ketones were productive substrates, acyclic ketones gave nuanced results that can be rationalized by stability of titanacyclobutane 4 (Law, et al., 2021; Straus, D. A., et al., Organometallics 1982, 1, 1658 (“Straus, et al., 1982”)). Thus, a series of methyl ketones where the steric interactions imposed by the neighboring alkyl group on 4 were systematically increased was evaluated. As the steric element (i.e., a phenyl group) was moved into close proximity to the Cp ligands in 4, the stability of this species decreased. For example, whereas benzylacetone gave azetidine 29 in 78% yield, phenylacetone furnished 30 in 54% yield. The major side product in this case was the 1,2-dibromide formed via electrophilic halogenation of the alkene generated in situ from Cp₂TiCH₂ and the parent ketone. Consistent with this trend, acetophenone furnished 31 (16% yield) alongside several side products derived from alkylation of the corresponding 1,2-dibromide and unreacted starting material. The impact of sterics was apparent using higher-order dialkyl ketones. Here, keeping the phenethyl group constant, 3-phenylpropanal gave 32 in 73% yield. However, as exemplified by products 33-36, unbranched alkyl groups (e.g. 33, R=Et) were problematic and branched alkyl groups (e.g. 36, R=i-Pr) emerged as limitations. Together, these results show that this method can be used to prepare 3-azetidines from relatively unhindered acyclic methyl ketones and aldehydes (Becker, M. R., et al., Nature Chem. 2020, 12, 898).

As noted in Scheme 4, aryl ketones were not fully consumed by reagent 3 at 0° C. Warming the reaction to accelerate the methylenation step was not feasible because titanacyclobutanes (4) decompose between 0-23° C. (Law, et al., 2021; Straus, et al., 1982). This issue was avoided by utilizing the corresponding styrenes (Scheme 5). In this case, the stoichiometry of 3 was reduced to 2 equiv.⁴¹ As outlined in reference Law, et al., 2021, 2 equiv of 3 are required to convert α-methylstyrene derivatives to titanacyclobutanes 4. Following this modification, using 1-methyleneindene as a substrate improved the yield of 28 to 63%. Similarly, a-methylstyrene facilitated access to 31 in 43% yield. This procedure was also compatible with other electron-deficient (hetero)arenes, as evidenced by 3-azetidines 37 and 38, which were produced in 55% and 81% yield, respectively, from the corresponding styrenes. As such, the reported strategy can be easily extended to 1,1-disubstituted alkenes, which are superior substrates to aromatic ketones.

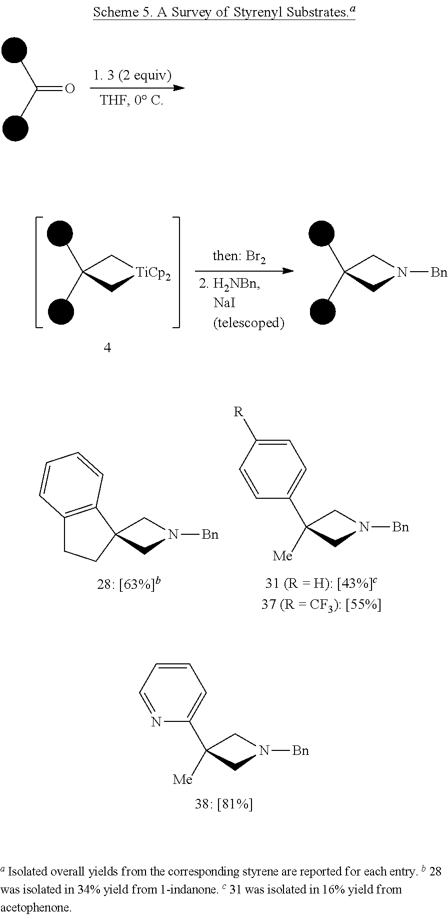

In summary, a direct strategy to prepare quaternary carbon centers from ketones was established. This approach exploits titanacyclobutanes generated in situ from C—O π-bonds via sequential carbonyl methylenation and alkene cyclometallation mediated by an excess of Tebbe's reagent (3). Halogenation of these transient organotitanium species provides functionalized all-carbon quaternary centers that are laborious to prepare any other way. The utility of this method was demonstrated via a versatile platform for the synthesis of azaspiro[3.n]alkanes and 3-azetidines. In this regard, the rather modest stability profile of titanacyclobutanes emerged as a limiting feature in some cases. Efforts to develop less sterically hindered titanium methylidene equivalents are on-going and will be reported in due course.

### Materials and Methods

General. Unless otherwise noted, reactions were conducted in oven-dried glassware (140° C.) under an atmosphere of nitrogen gas (N2) using anhydrous solvents. Tetrahydrofuran (THF), methylene chloride (CH2Cl2), diethyl ether (Et2O), toluene (PhMe), acetonitrile (MeCN), and dimethylformamide (DMF) were dried by passage through activated alumina using a solvent purification system (SPS). The preparation and titration of toluene solutions of Cp₂Ti(μ-Cl)(μ-CH₂)AlMe₂ (3) was carried out as described in detail previously (Law, J. L., et al., Angew. Chem. Int. Ed. 2021, 60, 14360-14364 “Law, et al., 2021”). Note: It is recommend to prepare reagent 3 before use. Bis(cyclopentadienyl)titanium dichloride was purchased from TCI and stored in a glovebox. Solutions of AlMe3 (2.0 M, PhMe) were purchased from Sigma Aldrich and used as received. Sodium sulfide, Dimethyl malonate, allylamine, benzylamine, and ethanethiol were purchased from and distilled prior to use. N-Boc-3-azetidinone (6) was purchased from Biosynth and used as received. Benzyl iodide were prepared according to known literature procedures (Hoang, C. T., et al., Org. Lett. 2007, 9, 2521-2524). The following substrates were purchased and used as received: N-Boc-3-pyrrolidinone, N-Boc-3-piperidone, N-Boc-4-piperidone, N-Boc-3-azacycoheptanone, 4-tetrahydropyranone, 1-indanone, cyclohexanone, cyclobutanone, 3,3-difluorocyclobutanone, benzylacetone, acetophenone, 3-phenylpropanal, and a-methylstyrene. All other starting materials were prepared according to literature procedures.

In general, one representative reaction and yield of the product is described in detail. All reported isolated yields are the average of duplicate or triplicate reactions, typically each within ±5% of the reported yield. Column chromatography was carried out using silica gel 60 (SiO2, 240-400 mesh) as stationary phase. Thin layer chromatography (TLC) was performed using pre-coated, glass-backed plates (SiO2, 60 PF254, 0.25 mm) and visualized by exposure to UV light (-254 nm) or by employing anisaldehyde, ninhydrin, and/or potassium permanganate staining. Unless otherwise stated, reactions were warmed in an oil bath to the indicated temperature and maintained using a mechanical stirrer equipped with a thermocouple.

1H NMR spectra were recorded at 400 MHz, 500 MHz or 600 MHz and are reported relative to the indicated deuterated solvent signals. Data for 1H NMR spectra are reported as follows: chemical shift (ppm), multiplicity, coupling constant (Hz), and integration. Splitting patterns are abbreviated as follows: singlet (s), doublet (d), triplet (t), quartet (q), quintet (quint), multiplet (m), broad (br), apparent (app), and combinations thereof. 13C NMR spectra were recorded at 100 MHz, 125 MHz, or 150 MHz. Data for 13C NMR spectra are reported in order of carbon multiplicity (C=quaternary, CH=methine, CH2=methylene, CH3=methyl) and chemical shift. Carbon multiplicity was established by a combination of DEPT135 or HMBC experiments. Reported melting points of solids are uncorrected. Mass spectra were collected on an LCT spectrometer utilizing direct analysis in real time (DART) ionization.

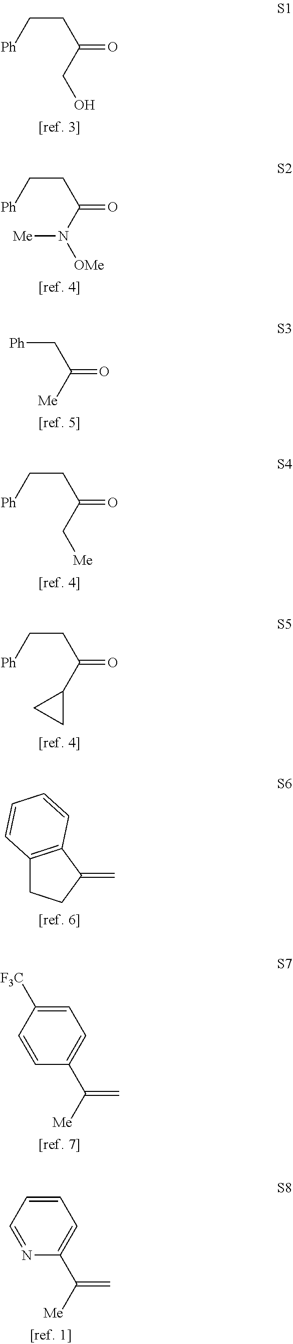

Starting materials prepared according to reported literature procedures: ref. 1 (see Law, et al., 2021), ref. 3 (see Krafft, et al., 1995), ref. 4 (see Lu, D, et al., Eur. J. Org. Chem. 2021, 4861-4864), ref. 5 (see PCT Publication No. WO 03087037A1 by Hagmann, W. K., et al.), ref. 6 (see Erden, I., et al., Eur. J. Org. Chem. 2017, 5147-5153), and ref. 7 (see Lux, M., et al., Org. Lett. 2020, 22, 3697-3701).

TABLE S1
Screening of Alternative Electrophiles

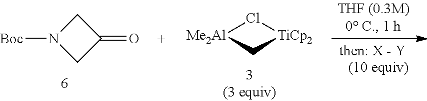

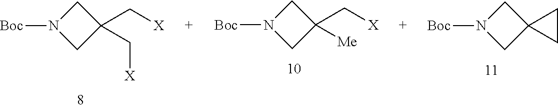

| | | | overall | | | 8:10:11 |
|---|---|---|---|---|---|---|
| | | | yield | | yield of | (¹H |
| entryª | X − Y | X | (%)^{b} | 8 | 8 (%)^{c} | NMR)^{d} |
| 1 | Br₂ | Br | 87 | 8a | 87 | >20:0:1 |
| 2 | NBS | Br | 9 | 8a | 3 | 3:1:7 |
| 3 | BDMS | Br | 15 | 8a | 10 | 4:2:1 |
| 4 | I₂ | I | 33 | 8b | 33 | 0:0:1 |
| 5 | NIS | I | 47 | 8b | 32 | 4:2:1 |
| 6 | ICl | I | 52 | 8b | 39 | 11:5:1 |
| 7 | NCS | Cl | 75 | 8c | 61 | 7:1:1 |
| 8 | DCDMH | C1 | 47 | 8c | 44 | 13:0:1 |
| 9 | Palau'Chlor | C1 | 53 | 8c | 38 | 3:5:2 |
| 10^{e} | TCCA | C1 | n.d.^{f} | 8c | n.d.^{f} | 4:12:1 |
| 11 | SO₂Cl₂ | CI | n.r.^{g} | 8c | n.r.^{g} | — |

^{a}A 1.0M solution of electrophile (X − Y) in CH₂Cl₂ was added after 1 h.
^{b}Combined isolated yield of 8, 10, and 11.
^{c}Isolated yield of 8 after purification by flash chromotography.
^{d}Determined by analyzing the ¹H NMR spectra of the unpurified reaction mixture.
^{e}THF was used in place of CH₂Cl₂.
^{f}n.d. = not determined.
^{g}n.r. = no desired reaction.

### Deuterium Labeling Experiments.

Mechanistic Hypothesis. Exposure of transient intermediate 4a to 2-(iodomethyl)naphthalene (13, 5 equiv) resulted in the formation of 14, d-14, and S9 after exposure to DCl. This inseparable mixture was observed along with d-10a, which was isolated in 74% yield. These observations are consistent with a radical mechanism, which might involve outer-shell electron transfer from 4a to 13 to generate a reactive intermediate akin to alkyl Ti(III) complex A (Scheme S1). Species of this type have been reported from oxidation of similar alkyl titanocenes (Burk, M. J., et al., J. Am. Chem. Soc. 1990, 112, 6133-6135). Abstraction of iodine from another equivalent of 13 would then give rise to B, which could react further with an exogenous benzyl radical to afford alkyl Ti(IV) species 12. Regardless of exact genesis of 12, the observation of d-14 and d-10a from deuterolysis (using DCl in D₂O) of the reaction mixture provides strong evidence for 12 as the main reactive intermediate.

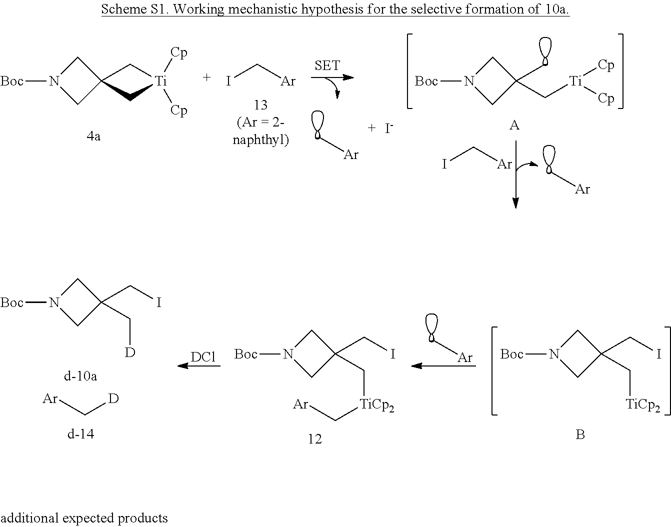

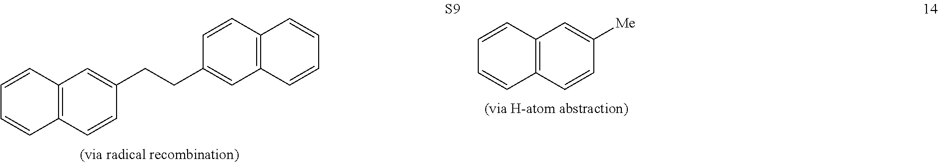

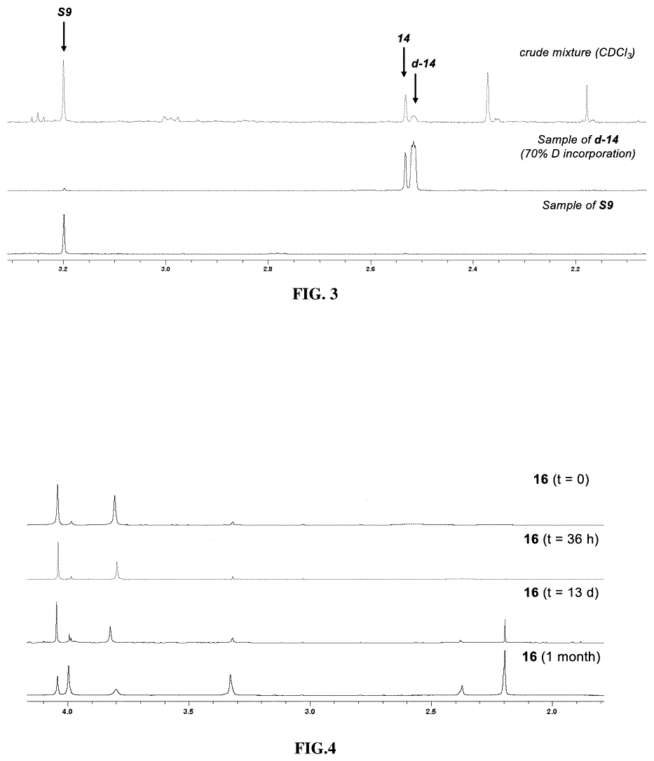

Results. To test Scheme 1, authentic samples of 14, d-14, and S9 were prepared. As shown in FIG. 3, all three of these expected byproducts were observed in the ¹H NMR spectrum of the unpurified reaction mixture following exposure to DCl. These species were inseparable, and notably, both 14 and d-14 are volatile.

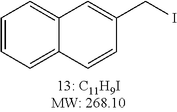 [SMILES: ICc1ccc2ccccc2c1]

2-(iodomethyl)naphthalene (13). A mixture of 2-(bromomethyl)naphthalene (5.53 g, 25.0 mmol) and sodium iodide (5.62 g, 27.5 mmol) in THF (25 mL) was stirred at r.t. (note: reaction was protected from light). After 12 h, the reaction mixture was transferred to a separatory funnel with H₂O (50 mL) and extracted with Et₂O (3×50 mL). The combined organic extracts were washed with 1 M aq. Na₂S₂O₃ (30 mL), dried over MgSO₄, filtered, and concentrated under reduced pressure to afford 13 (4.10 g, 15.3 mmol, 61%) as a yellow solid. No further purification was required: 1H NMR (600 MHz, CDCl3) 7.83-7.79 (m, 4H), 7.49-7.47 (m, 3H), 4.64 (s, 2H). All other characterization data was identical to previously reported values (Solladie-Cavallo, A., et al., A. J. Org. Chem. 1995, 60, 3494-3498).

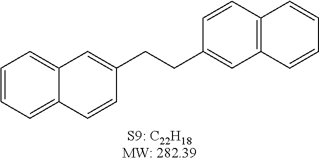 [SMILES: c1ccc2cc(CCc3ccc4ccccc4c3)ccc2c1]

1,2-bis-(2-naphthyl)ethane (S9). A mixture of 2-(bromomethyl)naphthalene (60 mg, 0.27 mmol) and THF (2.5 mL) was cooled to −78° C. After 0.25 h, n-BuLi (0.12 mL, 0.27 mmol, 2.3 M in hexanes) was added dropwise via syringe. The reaction mixture was maintained at −78° C. for 0.25 h, then treated with MeOH (0.06 mL). The resulting slurry was warmed to r.t. slowly. After 1 h, the resulting slurry was dried over MgSO₄, filtered, and concentrated under reduced pressure to provide S9 (24 mg, 0.17 mmol, 63% yield) as a colorless solid. No further purification was necessary: 1H NMR (600 MHz, CDCl3) 7.82-7.76 (m, 6H), 7.68 (s, 2H), 7.46-7.35 (m, 6H), 3.18 (s, 4H). All other characterization data was identical to previously reported values (Inaba, S., et al., J. Org. Chem. 1984, 49, 2093-2098).

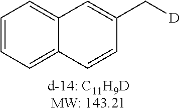 [SMILES: [2H]Cc1ccc2ccccc2c1]

2-(methyl-d)naphthalene (d-14). A mixture of LiAlD₄ (10.1 mg, 0.240 mmol) and THF (0.4 mL) was cooled to 0° C. After 10 min, a solution of 2-(bromomethyl)naphthalene (44.2 mg, 0.200 mmol) in THF (0.4 mL) was added dropwise via syringe. The resulting stirred slurry was allowed to warm to r.t. After 6 h, the mixture was treated successively with 1 mL Et₂O, 0.1 mL H₂O, 0.1 mL 15% aq. NaOH, and 0.2 mL H₂O. After 0.25 h, the resulting slurry was dried over MgSO₄, filtered, and concentrated under reduced pressure to provide d-14 (20.4 mg, 0.143 mmol, 71% yield) as a colorless solid: 1H NMR (400 MHz, CDCl3) (600 MHz, CDCl3) 7.80 (d, J=8.0 Hz, 1H), 7.75 (t, J=7.0 Hz, 2H) 7.62 (s, 1H), 7.45-7.39 (m, Hz, 2H), 7.32 (dd, J=8.4, 1.6 Hz, 1H), 2.50 (t, J=1.9 Hz, 2H). All other characterization data was identical to previously reported values (Yu, D. G., et al., J. Am. Chem. Soc. 2012, 134, 14638-14641). Deuterium incorporation (%) was determined by integration of distinct signals at 2.52 ppm (2-methylnaphthalene (Patra, T., et al., Chem. Commun. 2013, 49, 8362-8364)) and 2.50 ppm (d-14). This analysis revealed a 84% deuterium incorporation.

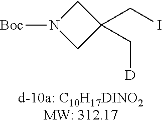 [SMILES: [2H]CC1(CI)CN(C(=O)OC(C)(C)C)C1]

tert-butyl 3-(iodomethyl)-3-(methyl-d)azetidine-1-carboxylate (d-10a). A solution of 3 (3.91 mL, 1.80 mmol, 0.46 M in PhMe) was transferred via syringe to a Schlenk tube and concentrated under reduced pressure (0.1 mBar, 0.5 h). The flask was backfilled with N₂ and the resulting residue was cooled to 0° C. After 10 min, a pre-cooled solution of 6 (103 mg, 0.600 mmol) in THF (2.0 mL) at 0° C. was added stream-wise via syringe. The resulting red solution was maintained at 0° C. for 1 h, then cooled to −78° C. After 0.25 h, a pre-cooled solution of 13 (804 mg, 3.00 mmol) in THF (3.0 mL) at −78° C. was added dropwise via syringe. The resulting solution was maintained at −78° C. for 0.25 h, then warmed to 0° C. After 12 h, the reaction septum was removed and the mixture was treated with DCl (1.00 mL, 4.00 mmol, 4.0 M in D₂O). The resulting slurry was allowed to warm to r.t. over 4 h, then transferred to a separatory funnel and washed with saturated aq. NaHCO₃ (3 mL). The aqueous layer was extracted with Et₂O (3×10 mL) and the combined organic extracts were dried over MgSO₄, filtered, and concentrated under reduced pressure. The resulting crude residue was purified by flash chromatography (SiO2, 6:1 hexanes/Et₂O) to afforded d-10a: (138 mg, 0.442 mmol, 74% yield) as a colorless oil: 1H NMR (600 MHz, CDCl3) δH3.68 (d, J=8.6 Hz, 2H), 3.60 (d, J=8.5 Hz, 2H), 3.39 (s, 2H), 1.44 (s, 9H), 1.37 (br s, 2H); HRMS-DART (m/z) [M+H]+ calculated for C10H18DINO2=313.0522; found 313.0508. Deuterium incorporation (%) was determined by integration of distinct signals at 1.39 ppm (14) and 1.37 ppm (d-14). This analysis revealed an 84% deuterium incorporation.

### Experimental Procedures and Characterization Data Starting Material Synthesis

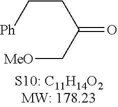 [SMILES: COCC(=O)CCc1ccccc1]

4-Methyl-3-phenyl-1-pentanone (S10). Following a modification of the procedure reported by Fievre (Krafft, M., et al., A. J. Org. Chem. 1995, 60, 5093-5101 (“Krafft, 1995”)), a suspension of S1 (500 mg, 3.04 mmol), methyl iodide (3.45 g, 24.3 mmol), and silver oxide (775 mg, 3.34 mmol), in MeCN (7.5 mL) was heated to 70° C. (note: reaction was protected from light). After 24 h, the reaction mixture was cooled to r.t., diluted with EtOAc (15 mL) and filtered over Celite. The filtrate was concentrated under reduced pressure and purified by flash chromatography (SiO2, 5:1 hex/EtOAc) to afford S9 (116 mg, 0.651 mmol, 21% yield) as a colorless oil: 1H NMR (600 MHz, CDCl3) δH7.28-7.18 (m, 5H), 3.96 (s, 2H), 3.38 (s, 3H), 2.93 (t, J=7.6 Hz, 2H), 2.77 (t, J=7.9 Hz, 2H). All other characterization was identical to reported values (Pace, V., et al., Chem. Commun. 2016, 52, 7584-7587).

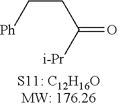 [SMILES: CC(C)C(=O)CCc1ccccc1]

4-Methyl-3-phenyl-1-pentanone (S11). A mixture of S2 (386 mg, 2.00 mmol) in THF (5.0 mL) was cooled to 0° C. and treated with i-PrMgCl (1.85 mL, 2.40 mmol, 1.30 M in THF). After 30 min, the mixture was allowed to warm to r.t. for 16 h, then treated with saturated aq. NH₄Cl (5 mL). The resulting slurry was transferred to a separatory funnel and extracted with CH₂Cl₂ (3×10 mL). The combined organic extracts were dried over MgSO₄, filtered, and concentrated under reduced pressure. The resulting crude residue was purified by flash chromatography (SiO2, 10:1 hex/EtOAc) to afford S10 (281 mg, 1.59 mmol, 80% yield) as a colorless oil: 1H NMR (400 MHz, CDCl3) δH 7.30-7.17 (m, 5H), 2.89 (t, J=5.4 Hz, 2H), 2.77 (t, J=7.6 Hz, 2H), 2.57 (t, J=7.7 Hz, 1H), 1.07 (m, J=7.0 Hz, 6H). All other characterization was identical to reported values (Mattson, M., et al., J. Org. Chem. 1996, 61, 6071-6074).

### Reactions of N-Boc-3-azetidenone (6) Shown in Table 1 and Scheme 2

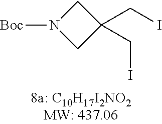 [SMILES: CC(C)(C)OC(=O)N1CC(CI)(CI)C1]

tert-Butyl 3,3-bis(iodomethyl)azetidine-1-carboxylate (8a). A solution of 3 (5.62 mL, 1.80 mmol, 0.31 M in PhMe) was added via syringe to a Schlenk tube and concentrated under reduced pressure (0.1 mBar, 0.5 h). The flask was backfilled with N₂ and the resulting red residue was cooled to 0° C. After 10 min, a pre-cooled solution of 6 (103 mg, 0.600 mmol) in THF (2.0 mL) at 0° C. was added stream-wise via syringe. The resulting red solution was maintained at 0° C. for 1 h, then cooled to −78° C. After 0.25 h, a pre-cooled solution of I₂ (1.52 g, 6.00 mmol) in THF (6.0 mL) at −78° C. was added dropwise via syringe. The resulting slurry was maintained at −78° C. for 0.25 h, then warmed to 0° C. After 0.75 h, the reaction septa was removed and the mixture was treated successively with SiO₂ (2.0 g), Et₂O (10 mL), and saturated aq. Na₂S2O₃ (0.35 mL). The resulting stirred slurry was allowed to warm to r.t. After 4 h, the resulting slurry was filtered and the resulting solution was concentrated under reduced pressure. The resulting residue was purified by flash chromatography (SiO2, 6:1 hexanes/Et₂O) afforded 8a (86 mg, 0.197 mmol, 33% yield) as a colorless oil: 1H NMR (400 MHz, CDCl3) δH 3.67 (s, 4H), 3.60 (s, 4H), 1.44 (s, 9H); 13C NMR (150 MHz, CDCl3) δ_{C} C: 156.1, 80.3, 39.0; CH2: 57.5, 16.0; CH3: 28.5; IR (thin-film): 2973, 2876, 1690, 1390, 1365 cm−1; HRMS-DART (m/z) [M+H]+ calculated for C10H18I2NO2=437.9427; found 437.9431.

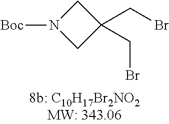 [SMILES: CC(C)(C)OC(=O)N1CC(CBr)(CBr)C1]

tert-Butyl 3,3-bis(bromomethyl)azetidine-1-carboxylate (8b). Following a modification of the procedure for 8a, the title compound was prepared from 3 (5.62 mL, 1.80 mmol, 0.31 M in PhMe) and 6 (103 mg, 0.600 mmol). The resulting red solution was maintained at 0° C. for 1 h, then cooled to −78° C. After 0.25 h, a pre-cooled solution of Br₂ (959 mg, 6.00 mmol) in THF (2.0 mL) at −78° C. was added dropwise via syringe. The resulting slurry was maintained at −78° C. for 0.25 h, then warmed to 0° C. After 0.75 h, the reaction septa was removed and the mixture was treated successively with SiO₂ (2.0 g), Et₂O (10 mL), and saturated aq. Na₂S₂O₃ (0.35 mL). The resulting stirred slurry was allowed to warm to r.t. After 4 h, the resulting slurry was filtered and the resulting solution was concentrated under reduced pressure. The resulting residue was purified by flash chromatography (SiO2, 6:1 hexanes/Et₂O) afforded 8b (179 mg, 0.523 mmol, 87% yield) as a yellow solid: mp=54-57° C.; 1H NMR (400 MHz, CDCl3) δH 3.75 (s, 4H), 3.74 (s, 4H), 1.44 (s, 9H); 13C NMR (150 MHz, CDCl3) δ_{C} C: 156.1, 80.3, 39.7; CH2: 57.0, 37.8; CH3: 28.4; IR (thin-film): 2975, 2881, 1690, 1391, 1366 cm−1; HRMS-DART (m/z) [M+H]+ calculated for C10H18Br2NO2=341.9704; found 341.9693.

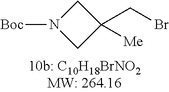 [SMILES: CC1(CBr)CN(C(=O)OC(C)(C)C)C1]

### tert-Butyl 3-(bromomethyl)-3-methylazetidine-1-carboxylate

(10b). An analytical sample of 10b was prepared by p-TLC (SiO2, 6:1 hexanes/Et₂O) from an early iteration of the procedure reported for 8b utilizing 10 equiv of NBS as the electrophile (Table S1, entry 3). Characterization data for this colorless oil is as follows: 1H NMR (500 MHz, CDCl3) δH 3.74 (d, J=8.6 Hz, 2H), 3.63 (d, J=8.7 Hz, 2H), 3.53 (s, 2H), 1.44 (s, 9H), 1.39 (s, 3H); 13C NMR (100 MHz, CDCl3) δ_{C} C: 156.5, 79.8, 35.3; CH2: 59.2, 42.2; CH3: 28.5, 23.6; IR (thin-film): 2965, 2879, 1690, 1391 cm−1; HRMS-DART (m/z) [M+H]+ calculated for C10H19BrNO2=264.0599; found 264.0599.

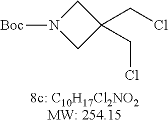 [SMILES: CC(C)(C)OC(=O)N1CC(CCl)(CCl)C1]

tert-Butyl 3,3-bis(chloromethyl)azetidine-1-carboxylate (8c). Following a modification of the procedure for 8a, the title compound was prepared from 3 (4.50 mL, 1.80 mmol, 0.40 M in PhMe) and 6 (103 mg, 0.600 mmol). The resulting red solution was maintained at 0° C. for 1 h, then cooled to −78° C. After 0.25 h, a pre-cooled solution of NCS (801 mg, 6.00 mmol) in THF (2.0 mL) at −78° C. was added dropwise via syringe. The resulting slurry was maintained at −78° C. for 0.25 h, then warmed to 0° C. After 0.75 h, the reaction septa was removed and the mixture was treated successively with SiO₂ (2.0 g), Et₂O (10 mL), and saturated aq. Na₂S₂O₃ (0.35 mL). The resulting stirred slurry was allowed to warm to r.t. After 4 h, the resulting slurry was filtered and the resulting solution was concentrated under reduced pressure. The resulting residue was purified by flash chromatography (SiO2, 6:1 hexanes/Et₂O) afforded 8c (94 mg, 0.370 mmol, 61% yield) as a colorless oil: 1H NMR (400 MHz, CDCl3) δH 3.83 (s, 4H), 3.74 (s, 4H), 1.45 (s, 9H); 13C NMR (150 MHz, CDCl3) δ_{C} C: 156.2, 80.3, 40.4; CH2: 55.5, 47.2; CH3: 28.4; IR (thin-film): 2976, 2884, 1701, 1393, 1365 cm−1; HRMS-DART (m/z) [M+H]+ calculated for C10H18Cl2NO2=254.0715; found 254.0698.

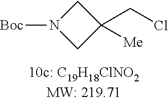 [SMILES: CC1(CCl)CN(C(=O)OC(C)(C)C)C1]

tert-Butyl 3-(chloromethyl)-3-methylazetidine-1-carboxylate (10c). An analytical sample of 10c was prepared by p-TLC (SiO2, 6:1 hexanes/Et₂O) from an early iteration of the procedure reported for 8c utilizing 10 equiv of Palau'Chlor as the electrophile (Table S1, entry 9). Characterization data for this colorless oil is as follows: 1H NMR (400 MHz, CDCl3) δH 3.76 (d, J=8.6 Hz, 2H), 3.62-3.60 (m, 4H), 1.44 (s, 9H), 1.36 (s, 3H); 13C NMR (150 MHz, CDCl3) δ_{C} C: 156.5, 79.8, 35.6; CH2: 58.2, 52.2; CH3: 28.5, 22.7; IR (thin-film): 2976, 2877, 1698, 1391, 1366 cm−1; HRMS-DART (m/z) [M+H]+ calculated for C10H19ClNO2=220.1104; found 220.1104.

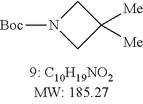 [SMILES: CC1(C)CN(C(=O)OC(C)(C)C)C1]

tert-Butyl 3,3-dimethylazetidine-1-carboxylate (9). A solution of 3 (4.09 mL, 1.80 mmol, 0.44 M in PhMe) was added via syringe to a Schlenk tube and concentrated under reduced pressure (0.1 mBar, 0.5 h). The flask was backfilled with N₂ and the resulting red residue was cooled to 0° C. After 10 min, a pre-cooled solution of 6 (103 mg, 0.600 mmol) in THF (2.0 mL) at 0° C. was added stream-wise via syringe. The resulting red solution was maintained at 0° C. for 1 h, then cooled to −78° C. After 0.25 h, the reaction exposed to air and treated with SiO₂ (2.0 g) and Et₂O (10 mL). The resulting stirred slurry was allowed to warm to r.t. After 4 h, the resulting slurry was filtered and the resulting solution was concentrated under reduced pressure. The resulting residue was purified by flash chromatography (SiO2, 6:1 hexanes/Et₂O) afforded 9 (103 mg, 0.556 mmol, 93% yield) as a colorless oil: 1H NMR (400 MHz, CDCl3) 3.61 (s, 4H), 1.46 (s, 9H), 1.26 (s, 6H). All other characterization data was identical to previously reported values (Law, et al., 2021).

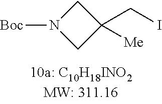 [SMILES: CC1(CI)CN(C(=O)OC(C)(C)C)C1]

tert-Butyl 3-(iodomethyl)-3-methylazetidine-1-carboxylate (10a). Following a modification of the procedure for d-10a, a solution of 3 (3.91 mL, 1.80 mmol, 0.46 M in PhMe) was added via syringe to a Schlenk tube and concentrated under reduced pressure (0.1 mBar, 0.5 h). The flask was backfilled with N₂ and the resulting red residue was cooled to 0° C. After 10 min, a pre-cooled solution of 6 (103 mg, 0.600 mmol) in THF (2.0 mL) at 0° C. was added stream-wise via syringe. The resulting red solution was maintained at 0° C. for 1 h, then cooled to −78° C. After 0.25 h, a pre-cooled solution of 13 (804 mg, 3.00 mmol) in THF (3.0 mL) at −78° C. was added dropwise via syringe. The resulting slurry was maintained at −78° C. for 0.25 h, then warmed to 0° C. After 12 h, the reaction septa was removed and the mixture was treated successively with SiO₂ (2.0 g) and Et₂O (10 mL). The resulting stirred slurry was allowed to warm to r.t. After 4 h, the resulting slurry was filtered and the resulting solution was concentrated under reduced pressure. The resulting residue was purified by flash chromatography (SiO2, 6:1 hexanes/Et₂O) afforded 10 (127 mg, 0.408 mmol, 68% yield) as a colorless oil: 1H NMR (600 MHz, CDCl3) δH 3.68 (d, J=8.6 Hz, 2H), 3.60 (d, J=8.6 Hz, 2H), 3.39 (s, 2H), 1.44 (s, 9H), 1.39 (s, 3H); 13C NMR (150 MHz, CDCl3) δ_{C} C: 156.2, 79.6, 34.9; CH2: 59.3, 18.4; CH3: 28.4, 25.3; IR (thin-film): 2879, 2695, 1690, 1391, 1365 cm−1; HRMS-DART (m/z) [M+H]+ calculated for C10H19INO2=312.0461; found 312.0461.

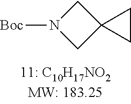 [SMILES: CC(C)(C)OC(=O)N1CC2(CC2)C1]

tert-Butyl 5-azaspiro[2.3]hexane-5-carboxylate (11). Following a modification of the procedure for 8a, the title compound was prepared from 3 (3.60 mL, 1.80 mmol, 0.50 M in PhMe) and 6 (103 mg, 0.600 mmol). The resulting red solution was maintained at 0° C. for 1 h, then cooled to −78° C. After 0.25 h, a pre-cooled solution of I₂ (457 mg, 1.80 mmol) in THF (2.0 mL) at −78° C. was added dropwise via syringe. The resulting slurry was maintained at −78° C. for 0.25 h, then warmed to 0° C. After 0.75 h, the reaction septa was removed and the mixture was treated successively with SiO₂ (2.0 g), Et₂O (10 mL), and saturated aq. Na₂S₂O₃ (0.35 mL). The resulting stirred slurry was allowed to warm to r.t. over 4 h, then filtered and filter cake was rinsed with Et₂O (2×5 mL). The resulting red solution was transferred to a separatory funnel with saturated aq. NaHCO₃ (3 mL) and extracted with Et₂O (2×5 mL). Ethanethiol (0.50 mL) and cesium carbonate (2.0 g) was added to the combined organic extracts and stirred at r.t. for two hours. The resulting slurry was stirred dried over MgSO₄, filtered, and concentrated under reduced pressure. The resulting crude residue was purified by flash chromatography (SiO2, 6:1 hexanes/Et₂O) to afford 11 (82 mg, 0.450 mmol, 75% yield) as a colorless oil: 1H NMR (600 MHz, CDCl3) H 3.98 (s, 4H), 1.45 (s, 9H), 0.60 (s, 4H); 13C NMR (150 MHz, CDCl3) δ_{C} C: 156.2, 79.4, 15.1; CH2: 57.2, 9.8; CH3: 28.6; IR (thin-film): 2977, 2876, 1698, 1477, 1356 cm−1; HRMS-DART (m/z) [M+H]+ calculated for C10H18NO2=184.1338; found 184.1327.

### Gram-scale Synthesis of tert-butyl 2,6-diazaspiro[3.3]heptane-2-carboxylate (16, Scheme 3)

Gram-scale synthesis of 8b. A flame-dried Schlenk tube (200 mL) equipped with a Teflon-coated stir bar was pumped into a glovebox. Under inert atmosphere (N2), the flask was charged with bis(cyclopentadienyl)titanium dichloride (5.96 g, 20.0 mmol), suspended in degassed PhMe (24 mL), protected from light, and treated with a solution of AlMe3 (22 mL, 22 mmol, 2.0 M in PhMe) at ambient temperature. The resulting dark red slurry was maintained at ambient temperature for 6 days in the glovebox. The resulting red solution was titrated with p-anisaldehyde as previously described, (Law, et al., 2021) to establish the concentration of 3 (0.40 M). The Schlenk tube was then removed from glove box and concentrated under reduced pressure (0.1 mBar, 2.5 h) at ambient temperature. The flask was backfilled with N₂ and the resulting red residue was cooled to 0° C. After 15 min, a pre-cooled solution of 6 (1.14 g, 6.67 mmol) in THF (22 mL) at 0° C. was added stream-wise via syringe. The resulting red solution was maintained at 0° C. for 1.5 h, then cooled to −78° C. After 0.25 h, a pre-cooled solution of Br₂ (10.6 g, 66.7 mmol) in CH₂Cl₂ (67.6 mL) at −78° C. was added dropwise via syringe over 0.25 h. The resulting slurry was maintained at −78° C. for 0.25 h, then warmed to 0° C. After 0.75 h, the was opened to atmosphere and the mixture was treated successively with SiO₂ (50 g), Et₂O (60 mL), and saturated aq. Na₂S203 (3.5 mL). The resulting stirred slurry was allowed to warm to r.t. over 4 h. The resulting slurry was filtered and the filter cake was rinsed with Et₂O (2×40 mL). The combined organic filtrate was concentrated under reduced pressure, and the resulting crude residue was filtered through a plug of SiO2 (15 g, 6:1 hexanes/Et₂O, 150 mL) to afford 8b (1.92 g, 5.59 mmol, 84% yield) as a yellow solid. No further purification was required.

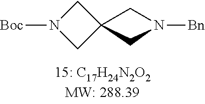

tert-Butyl 6-benzyl-2,6-diazaspiro[3.3]heptane-2-carboxylate (15). A suspension of 8b (2.75 g, 7.37 mmol), sodium iodide (2.20 g, 14.7 mmol), potassium carbonate (5.09 g, 36.8 mmol) and benzylamine (1.58 g, 14.7 mmol) in MeCN (25 mL) was heated to 80° C. After 20 h, the reaction mixture was cooled to r.t., diluted with Et₂O (70 mL), filtered, and concentrated under reduced pressure. The resulting residue was purified by flash chromatography (SiO2, 1:3 hexanes/Et₂O, 1% Et₃N) to afford 15 (1.86 g, 6.45 mmol, 88% yield) as a yellow oil: 1H NMR (600 MHz, CDCl3) δH 7.32-7.24 (m, 5H), 3.98 (s, 4H), 3.55 (s, 2H), 3.32 (s, 4H), 1.42 (s, 9H). 13C NMR (150 MHz, CDCl3) δ_{C} C: 156.2, 137.8, 79.6, 33.6; CH: 128.5, 128.5, 127.2; CH2: 64.4, 63.7, 59.7; CH3: 28.5; IR (thin-film): 3062, 3028, 2933, 2872, 1697, 1329 cm−1; HRMS-DART (m/z) [M+H]+ calculated for C17H25N₂02=289.1916; found 289.1915.

The following purification was required for reproducible results in the subsequent hydrogenation reaction. It might remove traces of S₈—or a similar inorganic impurity—which acts as a poison for Pd/C in the next step. A solution of 15 (1.80 g, 6.24 mmol) in Et₂O (10 mL) was transferred to separatory funnel and extracted with a 0.5 M solution of HCl (36 mL). The organic layer was discarded. The aqueous layer was washed with Et₂O (2×20 mL), then the pH was adjusted to ˜12 by addition of 1.0 M NaOH (20 mL). The resulting solution was extracted with CH₂Cl₂ (3×50 mL). The combined organic extracts were dried over MgSO₄, filtered, and concentrated under reduced pressure to provide 15 (1.78 g, 6.17 mmol, 99% recovery) as a yellow oil.

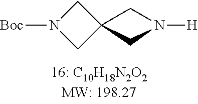

tert-Butyl 2,6-diazaspiro[3.3]heptane-2-carboxylate (16). A two-neck flask equipped with a two-way adapter was charged with palladium on carbon (1.72 g, 10% w/w) under positive pressure of N₂. The headspace was replaced with an atmosphere of H₂ (1 atm) via three cycles of evacuation and backfilling, and the flask was maintained under positive pressure of H₂ using a balloon. A solution of 15 (1.78 g, 6.17 mmol) in MeOH (20 mL) was added via syringe and the resulting stirred slurry was heated to 45° C. After 5 h, the reaction mixture was cooled to r.t., diluted with EtOAc (15 mL), and filtered over a pad of Celite. The resulting colorless solution was concentrated under reduced pressure to afford 16 (1.05 g, 5.32 mmol, 87% yield) as a colorless solid. No further purification was necessary: mp=126-129° C.; 1H NMR (400 MHz, CDCl3) δH 4.15 (s, 4H), 4.09 (s, 4H), 1.42 (s, 9H); 13C NMR (150 MHz, CDCl3) δ_{C} C: 155.8, 80.2, 35.4; CH2: 59.0, 55.4; CH3: 28.3; HRMS-DART (m/z) [M+H]+ calculated for C10H19N₂02=199.1446; found 199.1440.

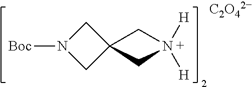

Oxalate Salt (for long-term storage): Following a modification of the procedure reported by Carreira (Burkhard, J., et al., Angew. Chem. Int. Ed. 2010, 49, 3524-3527 (“Burkhard, et al., 2010”)), a solution of 16 (1.05 g, 5.37 mmol) in Et₂O (82 mL) was treated with an 8.1 M (in Et₂O) solution of oxalic acid (143 mg, 2.68 mmol). The resulting solution was maintained at r.t. and a precipitate slowly formed. After 5 h, the resulting slurry was filtered to afford 6-(tert-butoxycarbonyl)-6-aza-2-azoniaspiro[3.3]heptane oxalate (1.16 g, 4.77 mmol, 89% yield) as a colorless solid. No further purification was required: 1H NMR (400 MHz, CD₃OD) δH 4.21 (s, 4H), 4.10 (s, 4H), 1.42 (s, 9H). All other characterization was identical to reported values (Burkhard, et al., 2010).

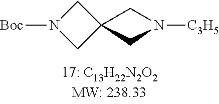

tert-butyl 6-allyl-2,6-diazaspiro[3.3]heptane-2-carboxylate (17). A suspension of 8b (68 mg, 0.20 mmol), sodium iodide (60 mg, 0.40 mmol), potassium carbonate (138 mg, 1.0 mmol) and allylamine (57 mg, 1.0 mmol) in MeCN (0.67 mL) was heated to 80° C. After 20 h, the reaction mixture was cooled to r.t., diluted with Et₂O (3 mL), filtered, and concentrated under reduced pressure. The resulting residue was purified by flash chromatography (SiO2, 3:1 hex/EtOAc, 1% Et₃N) to afford 17 (45 mg, 0.19 mmol, 94% yield) as a yellow oil: 1H NMR (400 MHz, CDCl3) δH 5.73 (ddt, J=16.6, 10.1, 6.0 Hz, 1H), 5.16 (d, J=17.2 Hz, 1H), 5.10 (d, J=10.2 Hz, 1H), 3.98 (s, 4H), 3.29 (s, 4H), 3.01 (d, J=6.1 Hz, 2H), 1.42 (s, 9H); 13C NMR (100 MHz, CDCl3) δ_{C} C: 156.1, 79.5, 33.6; CH: 134.0; CH2: 117.4, 64.2, 62.1, 59.5; CH3: 28.4; IR (thin-film): 3079, 3005, 2976, 2873, 1700, 1366 cm−1; HRMS-DART (m/z) [M+H]+ calculated for C13H23N₂O2=239.1759; found 239.1758.

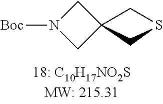

tert-butyl 2-thia-6-azaspiro[3.3]heptane-6-carboxylate (18). A suspension of 8b (68 mg, 0.20 mmol) and sodium sulfide (31 mg, 0.40 mmol) in MeCN (2.0 mL) and H₂O (0.22 mL) was heated to 50° C. After 3.5 h, the reaction mixture was cooled to r.t., transferred to a separatory funnel, and extracted with EtOAc (3×2 mL). The combined organic extracts were dried over MgSO₄, filtered, and concentrated under reduced pressure. The resulting crude residue was purified by flash chromatography (SiO2, 3:1 hexane/EtOAc) to afford 18 (36 mg, 0.17 mmol, 84% yield) as a colorless solid: 1H NMR (600 MHz, CDCl3) δH 3.95 (s, 4H), 3.33 (s, 4H), 1.43 (s, 9H). All other characterization was identical to reported values (Burkhard, J., et al., Org. Lett. 2008, 10, 3525-3526 (“Burkhard, et al., 2008”)).

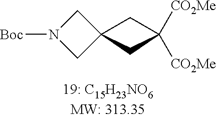

2-(tert-butyl) 6,6-dimethyl 2-azaspiro[3.3]heptane-2,6,6-tricarboxylate (19). A suspension of 8b (68 mg, 0.20 mmol), sodium iodide (60 mg, 0.40 mmol), potassium carbonate (138 mg, 1.0 mmol) and dimethyl malonate (106 mg, 0.80 mmol) in MeCN (0.67 mL) was heated to 80° C. After 20 h, the reaction mixture was cooled to r.t. and concentrated under reduced pressure. The resulting reside was purified by flash chromatography (SiO2, 5:1 hexane/Et2O) to afford 18 (40 mg, 0.13 mmol, 65% yield) as a colorless solid: mp=54-57° C.; 1H NMR (400 MHz, CDCl3) δH 3.92 (s, 4H), 3.75 (s, 6H), 2.72 (s, 4H), 1.42 (s, 9H); 13C NMR (150 MHz, CDCl3) δ_{C} C: 171.9, 156.1, 79.6, 48.2, 32.9; CH2: 61.4, 39.8; CH3: 53.0, 28.5; IR (thin-film): 2954, 2873, 2854, 1732, 1698, 1366 cm−1; HRMS-DART (m/z) [M+H]+ calculated for C15H24NO6=314.1604; found 314.1599.

Telescoped, 2-step synthesis of azaspiro[3.n]alkanes and 3-azetidines (Scheme 4). analytical samples of intermediate dibromides S12-S15 were prepared and fully characterized. This was generally unnecessary and no publication quality data was collected for most intermediate dibromides.

General Procedure A [For Cyclic Ketones]: A solution of 3 (0.40 M in PhMe, 3.0 equiv) was added via syringe to a Schlenk tube and concentrated under reduced pressure (0.1 mBar, 0.5 h). The flask was backfilled with N₂ and the resulting red residue was cooled to 0° C. After 10 min, a pre-cooled solution of ketone (1 equiv) in THF (0.3 M) at 0° C. was added stream-wise via syringe. The resulting red solution was maintained at 0° C. for 1 h, then cooled to −78° C. After 0.25 h, a pre-cooled solution of Br₂ (10 equiv) in CH₂Cl₂ (1.0 M) at −78° C. was added dropwise via syringe. The resulting slurry was maintained at −78° C. for 0.25 h, then warmed to 0° C. After 0.75 h, the reaction septum was removed and the mixture was treated successively with SiO₂ (3.3 g/mmol), Et₂O (15 mL/mmol), and saturated aq. Na₂S₂O₃ (0.60 mL/mmol). The resulting stirred slurry was allowed to warm to r.t. After 4 h, the slurry was filtered over a pad of SiO₂ (6.5 g/mmol, eluted with 90 mL/mmol of 5:1 hex/EtOAc) and the resulting solution was concentrated under reduced pressure. The resulting residue was digested with DMF (0.3 M) and treated with sodium iodide (2 equiv), potassium carbonate (5 equiv), and benzylamine (5 equiv). The resulting slurry was stirred rapidly and heated to 100° C. After 4 h, dibromide was fully consumed (as indicated by NMR). The reaction mixture was cooled to r.t., diluted with Et₂O (15 mL/mmol) and filtered. The filtrate was concentrated under reduced pressure and the resulting crude reside was purified as indicated.

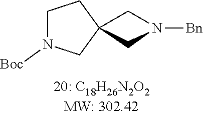

tert-butyl 2-benzyl-2,6-diazaspiro[3.4]octane-6-carboxylate (20). Following General Procedure A, the title compound was prepared from N-Boc-pyrrolidinone (111 mg, 0.600 mmol). Purification by flash chromatography (SiO2, 1:3 hexanes/Et₂O, 1% Et₃N) afforded 20 (116 mg, 0.384 mmol, 64% yield) as a yellow oil. At r.t., compound 20 exists as a mixture of rotational isomers (rotamers) around the carbamate bond in CDCl₃: 1H NMR (600 MHz, CDCl3) rotamers 1 & 2 are indistinguishable by 1H NMR: δH 7.33-7.27 (m, 5H), 3.67 (br s, 2H), 3.43-3.22 (m, 6H), 2.09 (br s, 1H), 2.01 (br s, 1H), 1.45 (s, 9H); 13C NMR (150 MHz, CDCl3) rotamer 1: δ_{C} C: 154.7, 138.1, 79.3, 41.1; CH: 128.6, 128.4, 127.2; CH2: 63.8, 55.9, 44.9, 36.2; CH3: 28.6; rotamer 2: δ_{C} C: 154.7, 138.1, 79.3, 40.3; CH: 128.6, 128.4, 127.2; CH2: 63.6, 55.2, 44.4, 35.5; CH3: 28.6; HRMS-DART (m/z) [M+H]+ calculated for C18H27N₂O2=303.2072; found 303.2051.

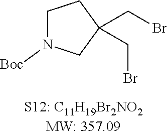 [SMILES: CC(C)(C)OC(=O)N1CCC(CBr)(CBr)C1]

tert-butyl 3,3-bis(bromomethyl)pyrrolidine-1-carboxylate (S12). An analytical sample of S12 was prepared by flash chromatography (SiO₂, 6:1 hexanes/Et₂O): ¹H NMR (400 MHz, CDCl3) rotamers 1 & 2 are indistinguishable by 1H NMR δH 3.58 (s, 4H), 3.48-3.37 (m, 4H), 1.99 (t, J=6.4 Hz, 2H), 1.46 (s, 9H); 13C NMR (100 MHz, CDCl3) rotamer 1: δ_{C} C: 154.4, 80.0, 48.0; CH2: 54.1, 44.8, 38.1. 33.8; CH3: 28.4; rotamer 2: δ_{C} C: 154.4, 80.0, 47.1; CH2: 54.1, 44.5, 38.1. 33.2; CH3: 28.4; IR (thin-film): 2974, 2873, 1600, 1396, 1365 cm−1; HRMS-DART (m/z) [M+H]+ calculated for C11H20Br₂NO2=355.9861; found 355.9842.

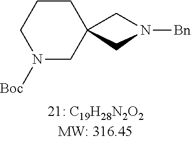

tert-butyl 2-benzyl-2,6-diazaspiro[3.5]nonane-6-carboxylate (21). Following General Procedure A, the title compound was prepared from N-Boc-3-piperidinone (120 mg, 0.600 mmol). Purification by flash chromatography (SiO2, 1:3 hexanes/Et₂O, 1% Et₃N) afforded 21 (120 mg, 0.379 mmol, 63% yield) as a yellow oil. At r.t., compound 21 exists as a mixture of rotational isomers (rotamers) around the carbamate bond in CDCl₃: 1H NMR (600 MHz, CDCl3) rotamer 1 & 2 are indistinguishable by 1H NMR δH 7.31-7.23 (m, 5H), 3.66 (br s, 2H), 3.49 (br s, 2H), 3.30 (br s, 2H), 3.08 (br s, 2H), 2.88 (br s, 4H), 1.72 (br s, 2H), 1.46 (s, 11H); 13C NMR (150 MHz, CDCl3) rotamer 1: δ_{C} C: 155.2, 138.5, 79.5, 35.6; CH: 128.4, 128.3, 127.0; CH2: 63.6. 62.6, 53.0, 44.5, 35.2, 22.5; CH3: 28.6; rotamer 2: δ_{C} C: 155.2, 138.5, 79.5, 35.6; CH: 128.4, 128.3, 127.0; CH2: 63.6. 62.6, 52.3, 43.7, 35.2, 22.5; CH3: 28.6; HRMS-DART (m/z) [M+H]+ calculated for C19H29N₂O2=317.2229; found 317.2285.

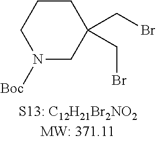 [SMILES: CC(C)(C)OC(=O)N1CCCC(CBr)(CBr)C1]

tert-butyl 3,3-bis(bromomethyl)piperidine-1-carboxylate (S13). An analytical sample of S13 was prepared by flash chromatography (SiO₂, 6:1 hexanes/Et₂O): 1H NMR (600 MHz, CDCl3): mp=67-69° C.; 1H NMR (600 MHz, CDCl3) δH 3.47 (s, 4H), 3.43 (br. s, 2H), 3.38 (br. s, 2H), 1.68 (br. s, 2H), 1.56 (br. s, 2H), 1.47 (s, 9H); 13C NMR (150 MHz, CDCl3) δ_{C} C: 154.4, 79.9, 44.5; CH2: 49.9, 38.4, 43.4 31.5, 20.9; CH3: 28.3; HRMS-DART (m/z) [M+H]+ calculated for C12H22Br₂NO2=370.0017; found 369.9987.

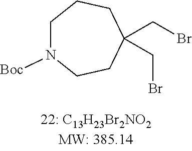 [SMILES: CC(C)(C)OC(=O)N1CCCC(CBr)(CBr)CC1]

tert-butyl 4,4-bis(bromomethyl)azepane-1-carboxylate (22). A solution of 3 (5.00 mL, 1.80 mmol, 0.36 M in PhMe) was added via syringe to a Schlenk tube and concentrated under reduced pressure (0.1 mBar, 0.5 h). The flask was backfilled with N₂ and the resulting red residue was cooled to 0° C. After 10 min, a pre-cooled solution of N-Boc-hexahydro-1H-azepin-4-one (128 mg, 0.600 mmol) in THF (2.0 mL) at 0° C. was added stream-wise via syringe. The resulting red solution was maintained at 0° C. for 1 h, then cooled to −78° C. After 0.25 h, a pre-cooled solution of Br₂ (959 mg, 6.00 mmol) in CH₂Cl₂ (6.0 mL) at −78° C. was added dropwise via syringe. The resulting slurry was maintained at −78° C. for 0.25 h, then warmed to 0° C. After 0.75 h, the reaction septum was removed and the mixture was treated successively with SiO₂ (2.0 g), Et₂O (10 mL), and saturated aq. Na₂S₂O₃ (0.35 mL). The resulting stirred slurry was allowed to warm to r.t. After 4 h, the slurry was filtered and the filtrate was concentrated under reduced pressure. The resulting crude residue was purified by flash chromatography (SiO2, 6:1 hexanes/Et₂O) to afford 22 (147 mg, 0.382 mmol, 64% yield) as a colorless oil. At r.t., compound 22 exists as a mixture of rotational isomers (rotamers) around the carbamate bond in CDCl₃: 1H NMR (400 MHz, CDCl3) rotamers 1 & 2 are indistinguishable by 1H NMR δH 3.47 (s, 4H), 3.46-3.36 (m, 4H), 1.83 (t, J=5.5 Hz, 1H), 1.78 (t, J=5.8 Hz, 1H), 1.70 (br s, 4H), 1.47 (s, 9H); 13C NMR (100 MHz, CDCl3) rotamer 1: δ_{C} C: 155.3, 79.8, 40.4; CH2: 46.0, 42.4, 41.5, 34.9, 33.6, 23.7; CH3: 28.5; rotamer 2: δ_{C} C: 155.3, 79.8, 40.4; CH2: 45.3, 41.7, 41.3, 34.7, 33.6, 23.4; CH3: 28.5; IR (thin-film): 2974, 2869, 1685, 1451, 1365 cm−1; HRMS-DART (m/z) [M+H]+ calculated for C13H24Br₂NO2=384.0174; found 384.0192.

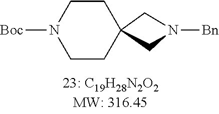

tert-butyl 2-benzyl-2,7-diazaspiro[3.5]nonane-7-carboxylate (23). Following General Procedure A, the title compound was prepared from N-Boc-4-piperidinone (120 mg, 0.600 mmol). Purification by flash chromatography (SiO2, 1:3 hexanes/Et₂O, 1% Et₃N) afforded 23 (126 mg, 0.398 mmol, 66% yield) as a yellow oil. 1H NMR (500 MHz, CDCl3) δH 7.32-7.23 (m, 5H), 3.64 (s, 2H), 3.31 (t, J=5.6 Hz, 4H), 3.04 (s, 4H), 1.69 (t, J=5.7 Hz, 4H), 1.44 (s, 9H); 13C NMR (125 MHz, CDCl3) δ_{C} C: 154.9, 138.3, 79.4, 34.5; CH: 128.5, 128.4, 127.0; CH2: 64.0. 63.8, 41.4, 36.0; CH3: 28.5; HRMS-DART (m/z) [M+H]+ calculated for C19H29N₂O2=317.2229; found 317.2225.

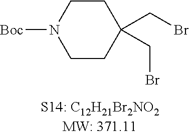 [SMILES: CC(C)(C)OC(=O)N1CCC(CBr)(CBr)CC1]

tert-butyl 4,4-bis(bromomethyl)piperidine-1-carboxylate (S14). An analytical sample of S14 was prepared by flash chromatography (SiO₂, 6:1 hexanes/Et₂O): mp=58-60° C.; 1H NMR (400 MHz, CDCl3) δH 3.52 (s, 4H), 3.39 (t, J=5.8 Hz, 4H), 1.66 (t, J=5.9 Hz, 4H), 1.46 (s, 9H); 13C NMR (150 MHz, CDCl3) δ_{C} C: 154.7, 79.9, 36.7; CH2: 39.9, 39.4, 32.2; CH3: 28.5; IR (thin-film): 2974, 2862, 1685, 1418, 1364 cm−1; HRMS-DART (m/z) [M+H]+ calculated for C12H22Br₂NO2=370.0017; found 370.0020.

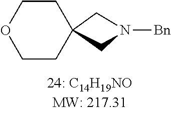

2-benzyl-7-oxa-2-azaspiro[3.5]nonane (24). Following General Procedure A, the title compound was prepared from tetrahydro-4H-pyran-4-one (60 mg, 0.600 mmol). Purification by flash chromatography (SiO2, 1:2 hexanes/Et₂O, 1% Et₃N) afforded 24 (0.095 g, 0.437 mmol, 73% yield) as a yellow oil. 1H NMR (500 MHz, CDCl3) δH 7.32-7.25 (m, 5H), 3.66 (s, 2H), 3.58 (t, J=5.2 Hz, 4H), 3.08 (s, 4H), 1.76 (t, J=5.3 Hz, 4H); 13C NMR (125 MHz, CDCl3) δ_{C} C: 138.4, 33.7; CH: 128.4, 128.3, 127.0; CH2: 65.1, 64.5, 63.8, 37.0; HRMS-DART (m/z) [M+H]+ calculated for C14H20NO=218.1545; found 218.1531.

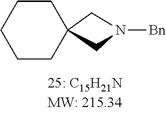

2-benzyl-2-azaspiro[3.5]nonane (25). Following General Procedure A, the title compound was prepared from cyclohexanone (59 mg, 0.600 mmol). Purification by flash chromatography (SiO2, 1:1 hexanes/Et₂O, 1% Et₃N) afforded 25 (80 mg, 0.371 mmol, 62% yield) as a yellow oil. 1H NMR (600 MHz, CDCl3) δH 7.43-7.24 (m, 5H), 3.67 (s, 2H), 3.03 (br. s, 4H), 1.62 (s, 4H), 1.39 (br. s, 6H); 13C NMR (125 MHz, CDCl3) δ_{C} C: 127.2, 36.1; CH: 128.7, 128.4; CH2: 65.0, 63.7, 37.2, 25.9, 23.3; HRMS-DART (m/z) [M+H]+ calculated for C15H22N=216.1752; found 216.1755.

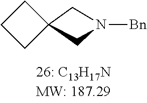

2-benzyl-2-azaspiro[3.3]heptane (26). Following General Procedure A, the title compound was prepared from cyclobutanone (42 mg, 0.600 mmol). Purification by flash chromatography (SiO2, 1:1 hexanes/Et₂O, 1% Et₃N) afforded 26 (65 mg, 0.35 mmol, 58% yield) as a yellow oil.

1H NMR (600 MHz, CDCl3) δH 7.28-7.23 (m, 5H), 3.57 (s, 2H), 3.21 (s, 4H), 2.10 (t, J=7.6 Hz, 4H), 1.79 (quint, J=7.6 Hz, 2H); 13C NMR (150 MHz, CDCl3) δ_{C} C: 138.5, 39.2; CH: 128.6, 128.4, 127.0; CH2: 67.0, 64.0, 33.2, 16.9; HRMS-DART (m/z) [M+H]+ calculated for C13H18N=188.1439; found 188.1431.

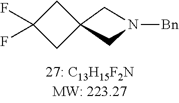

2-benzyl-6,6-difluoro-2-azaspiro[3.3]heptane (27). Following General Procedure A, the title compound was prepared from 3,3-difluorocyclobutanone (64 mg, 0.600 mmol). Purification by flash chromatography (SiO2, 1:1 hexanes/Et₂O, 1% Et₃N) afforded 27 (76 mg, 0.340 mmol, 57% yield) as a yellow oil. 1H NMR (600 MHz, CDCl3) δH 7.32-7.23 (m, 5H), 3.58 (s, 2H), 3.29 (s, 4H), 2.68 (t, J=12.2 Hz, 4H) 13C NMR (150 MHz, CDCl3) δ_{C} C: 137.9, 119.17 (t, J=273.5 Hz), 28.2 (t, J=10.5 Hz); CH: 128.6, 128.5, 127.2; CH2: 65.3, 63.8, 45.7 (t, J=23.0 Hz); 19F NMR (564 MHz, CDCl3) δ_{F} −91.9 (s, 2F); HRMS-DART (m/z) [M+H]+ calculated for C13H16NF₂=224.1251; found 224.1243.

General Procedure B [For Acyclic Ketones and Aldehydes]: A solution of 3 (0.40 M in PhMe, 3.0 equiv) was added via syringe to a Schlenk tube and concentrated under reduced pressure (0.1 mBar, 0.5 h). The flask was backfilled with N₂ and the resulting red residue was cooled to −10° C. After 10 min, a pre-cooled solution of acyclic ketone or aldehyde (1 equiv) in THF (0.3 M) at 0° C. was added stream-wise via syringe. The resulting red solution was maintained at −10° C. for 4 h, then cooled to −78° C. After 0.25 h, a pre-cooled solution of Br₂ (10 equiv) in CH₂Cl₂ (1.0 M) at −78° C. was added dropwise via syringe. The resulting slurry was maintained at −78° C. for 0.25 h, then warmed to 0° C. After 0.75 h, the reaction septum was removed and the mixture was treated successively with SiO₂ (3.3 g/mmol), Et₂O (15 mL/mmol), and saturated aq. Na₂S₂O₃ (0.60 mL/mmol). The resulting stirred slurry was allowed to warm to r.t. After 4 h, the slurry was filtered over a pad of SiO₂ (6.5 g/mmol, eluted with 90 mL/mmol of 10:1 hex/EtOAc) and the resulting solution was concentrated under reduced pressure. The resulting crude residue was digested with DMF (0.3 M) and treated with sodium iodide (2 equiv), potassium carbonate (5 equiv), and benzylamine (10 equiv). The resulting slurry was heated to 100° C. After the consumption of the dibromide (as indicated by ¹H NMR), the resulting reaction mixture was cooled to r.t., diluted with Et₂O (15 mL/mmol) and filtered. The filtrate was concentrated under reduced pressure and the resulting crude reside was then purified as indicated.

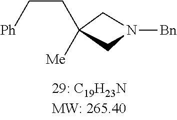

1-benzyl-3-methyl-3-phenethylazetidine (29). Following General Procedure B, the title compound was prepared from benzylacetone (89 mg, 0.600 mmol). Purification by flash chromatography (SiO2, 2:1 hexanes/EtOAc) afforded 29 (124 mg, 0.467 mmol, 78% yield) as a colorless oil: 1H NMR (600 MHz, CDCl3) δH 7.32-7.17 (m, 10H), 3.64 (s, 2H), 3.03 (br. s, 2H), 2.56 (app t, J=8.5 Hz, 2H), 1.86 (app t, J=8.7 Hz, 2H), 1.32 (s, 3H); 13C NMR (150 MHz, CDCl3) δ_{C} C: 142.7, 138.7, 34.9; CH: 128.4, 128.3, 126.9, 125.8; CH2: 65.7, 63.8, 42.9, 31.2; CH3: 24.8; HRMS-DART (m/z) [M+H]+ calculated for C19H24N=266.1909; found 266.1902.

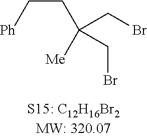 [SMILES: CC(CBr)(CBr)CCc1ccccc1]

(4-bromo-3-(bromomethyl)-3-methylbutyl)benzene (S15) An analytical sample of S15 was prepared by flash chromatography (SiO2, 20:1 hexanes/Et₂O): 1H NMR (600 MHz, CDCl3) δH 7.31-7.20 (m, 5H), 3.48 (dd, J=7.6 Hz, 4H), 2.59 (app t, J=7.9 Hz, 2H), 1.75 (app t, J=8.6 Hz, 2H) 1.22 (s, 3H); 13C NMR (150 MHz, CDCl3) δ_{C} C: 141.6, 38.4; CH: 128.6, 128.4, 126.2; CH2: 41.6, 40.0, 30.4; CH3: 21.9; HRMS-DART (m/z) [M+H]+ calculated for C12H17Br2=318.9697; found 318.9723.

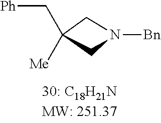

1,3-dibenzyl-3-methylazetidine (30). Following General Procedure B, the title compound was prepared from S3 (81 mg, 0.600 mmol). Purification by flash chromatography (SiO2, 2:1 hexanes/EtOAc) afforded 30 (86 mg, 0.342 mmol, 57% yield) as a colorless oil: 1H NMR (600 MHz, CDCl3) δH 7.32-7.13 (m, 10H), 3.63 (s, 2H), 3.18 (d, J=7.0 Hz, 2H), 3.00 (d, J=7.0 Hz, 2H) 2.86 (s, 2H), 1.20 (s, 3H); 13C NMR (150 MHz, CDCl3) δ_{C} C: 139.2, 138.6, 35.8; CH: 129.8, 128.5, 128.4, 128.2, 127.0, 126.2; CH2: 65.4, 63.7, 46.6; CH3: 25.0; HRMS-DART (m/z) [M+H]+ calculated for C18H22N=252.1752; found 252.1756.

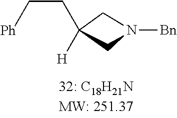

1-benzyl-3-phenethylazetidine (32). Following General Procedure B, the title compound was prepared from 3-phenylpropanal (81 mg, 0.600 mmol). Purification by flash chromatography (SiO2, 2:1 hexanes/EtOAc) afforded 32 (110 mg, 0.438 mmol, 73% yield) as a colorless oil: 1H NMR (600 MHz, CDCl3) δH 7.32-7.13 (m, 10H), 3.60 (s, 2H), 3.47 (t, J=7.4 Hz, 2H), 2.81 (t, J=6.8 Hz, 2H), 2.55-2.50 (m, 3H), 1.85 (q, J=7.7 Hz, 1H); 13C NMR (150 MHz, CDCl3) δ_{C} C: 142.1, 138.5; CH: 128.6, 128.5, 128.4, 128.4, 127.0, 125.9, 30.9; CH2: 64.1, 60.7, 36.4, 33.8; HRMS-DART (m/z) [M+H]+ calculated for C18H22N=252.1752; found 252.1750.

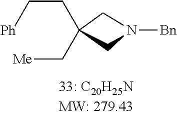

1-benzyl-3-ethyl-3-phenethylazetidine (33). Following General Procedure B, the title compound was prepared from S4 (97 mg, 0.600 mmol). Purification by flash chromatography (SiO2, 2:1 hexanes/EtOAc) afforded 33 (58 mg, 0.21 mmol, 35% yield) as a colorless oil: 1H NMR (600 MHz, CDCl3) δH 7.30-7.19 (m, 10H), 3.68 (br. s, 2H), 3.04 (br. s, 4H), 2.52 (app. t, J=8.5 Hz, 2H), 1.89 (app. t, J=8.6 Hz, 2H), 1.73 (q, J=7.4 Hz, 2H), 0.87 (t, J=7.4 Hz, 3H); 13C NMR (150 MHz, CDCl3) δ_{C} C: 142.8, 138.7, 38.3; CH: 128.5, 128.5, 128.4, 128.4, 127.0, 125.8; CH2: 64.0, 63.7, 38.8, 30.8, 29.6; CH3: 8.5; HRMS-DART (m/z) [M+H]+ calculated for C20H26N=280.2065; found 280.2065.

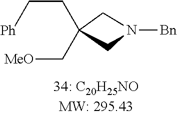

1-benzyl-3-(methoxymethyl)-3-phenethylazetidine (34). Following General Procedure B, the title compound was prepared from S10 (107 mg, 0.600 mmol). Purification by flash chromatography (SiO2, 1:1 hexanes/EtOAc) afforded 34 (42 mg, 0.14 mmol, 24% yield) as a colorless oil: 1H NMR (600 MHz, CDCl3) δH 7.32-7.16 (m, 10H), 3.66 (br. s, 2H), 3.53 (s, 2H), 3.39 (s, 3H), 3.19 (br. s, 2H), 3.01 (br. s, 2H), 2.57 (br. s, 2H), 2.57 (app. t, J=8.4 Hz, 2H), 1.96 (app. t, J=8.6 Hz, 2H); 13C NMR (150 MHz, CDCl3) δ_{C} C: 142.6, 138.5 (br.), 38.8; CH: 128.6, 128.5, 128.5, 128.4, 127.1, 125.9; CH2: 63.4, 61.5, 59.4, 37.8, 30.9; CH3: 77.1; HRMS-DART (m/z) [M+H]+ calculated for C20H26NO=296.2014; found 296.2011.

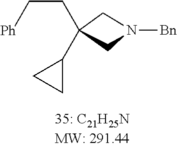

1-benzyl-3-cyclopropyl-3-phenethylazetidine (35). Following General Procedure B, the title compound was prepared from S5 (105 mg, 0.600 mmol). Purification by flash chromatography (SiO2, 2:1 hexanes/EtOAc) afforded 35 (15.5 mg, 0.0532 mmol, 9% yield) as a colorless oil: 1H NMR (600 MHz, CDCl3) δH 7.31-7.17 (m, 10H), 3.62 (br. s, 2H), 2.98 (d, J=6.3 Hz, 2H), 2.84 (d, J=6.3 Hz, 2H), 2.70 (app. t, J=8.5 Hz, 2H), 2.05 (app. t, J=8.6 Hz, 2H), 0.92-0.86 (m, 2H), 0.52-0.49 (m, 2H), 0.35-0.32 (m, 2H); 13C NMR (150 MHz, CDCl3) δ_{C} C: 143.0, 127.0, 38.3; CH: 128.5, 128.5, 128.4, 125.8, 16.1; CH2: 63.2, 61.1, 43.3, 31.3, 1.7; HRMS-DART (m/z) [M+H]+ calculated for C21H26N=292.2065; found 292.2059.

Telescoped synthesis of 3-azetidines from styrenes (Scheme 5). analytical samples of intermediate dibromides S16-S18 were prepared and fully characterized.

General Procedure C [For styrenyl substrates]: A solution of 3 (0.40 M in PhMe, 2.0 equiv) was added via syringe to a Schlenk tube and concentrated under reduced pressure (0.1 mBar, 0.5 h). The flask was backfilled with N₂ and the resulting red residue was cooled to −10° C. After 10 min, a pre-cooled solution of styrene (1 equiv) in THF (0.3 M) at 0° C. was added stream-wise via syringe. The resulting red solution was maintained at −10° C. for 3 h, then cooled to −78° C. After 0.25 h, a pre-cooled solution of Br₂ (10 equiv) in CH₂Cl₂ (1.0 M) at −78° C. was added dropwise via syringe. The resulting slurry was maintained at −78° C. for 0.25 h, then warmed to 0° C. After 0.75 h, the reaction septa was removed and the mixture was treated successively with SiO₂ (3.3 g/mmol), Et₂O (15 mL/mmol), and saturated aq. Na₂S₂O₃ (0.60 mL/mmol). The resulting stirred slurry was allowed to warm to r.t. After 4 h, the resulting slurry was filtered over a pad of SiO₂ (6.5 g/mmol, eluted with 90 mL/mmol of 10:1 hex/EtOAc) and the resulting solution was concentrated under reduced pressure. The crude residue was diluted with DMF (0.3 M) and treated with sodium iodide (2 equiv), potassium carbonate (5 equiv), and benzyl amine (10 equiv). The resulting slurry was heated to 100° C. After the consumption of the dibromide (as indicated by ¹H NMR), the resulting reaction mixture was cooled to r.t., diluted with Et₂O (15 mL/mmol) and filtered. The filtrate was concentrated under reduced pressure and the resulting crude reside was then purified as indicated.

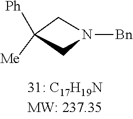

1-Benzyl-3-methyl-3-phenylazetidine (31). Following General Procedure C, the title compound was prepared from a-methylstyrene (72 mg, 0.600 mmol). Purification by flash chromatography (SiO2, 3:1 hexanes/EtOAc) afforded 31 (62 mg, 0.261 mmol, 43% yield) as a colorless oil: 1H NMR (400 MHz, CDCl3) δH 7.34-7.12 (m, 10H), 3.68 (s, 2H), 3.51 (d, J=7.1 Hz, 2H), 3.39 (d, J=7.1 Hz, 2H), 1.66 (s, 3H); 13C NMR (125 MHz, CDCl3) δ_{C} C: 149.3, 138.5, 39.2; CH: 128.6, 128.5, 128.4, 127.1, 125.9, 125.2; CH2: 66.1, 63.7; CH3: 29.5; HRMS-DART (m/z) [M+H]+ calculated for C17H20N=238.1596; found 238.1600.

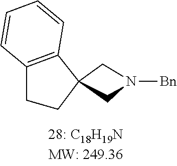

1-Benzyl-2′,3′-dihydrospiro[azetidine-3,1′-indene] (28). Following General Procedure C, the title compound was prepared from S6 (78 mg, 0.600 mmol). Purification by flash chromatography (SiO2, 2:1 hexanes/EtOAc) afforded 28 (94 mg, 0.378 mmol, 63% yield) as a yellow oil: 1H NMR (600 MHz, CDCl3) δH 7.55 (d, J=7.5 Hz, 1H) 7.36-7.17 (m, 8H), 3.73 (s, 2H), 3.44 (d, J=7.4 Hz, 2H), 3.38 (d, J=7.4 Hz, 2H), 2.88 (t, J=7.3 Hz, 2H), 2.38 (t, J=7.3 Hz, 2H); 13C NMR (150 MHz, CDCl3) δ_{C} C: 147.6, 143.7, 138.5, 46.8; CH: 128.6, 128.4, 127.1, 127.0, 126.8, 124.4, 123.2; CH2: 67.0, 63.7, 38.7, 30.7; HRMS-DART (m/z) [M+H]+ calculated for C18H20N=250.1596; found 250.1595.

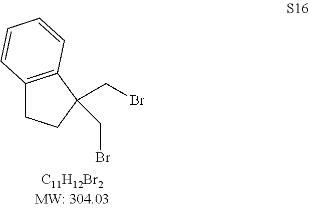 [SMILES: BrCC1(CBr)CCc2ccccc21]

1,1-Bis(bromomethyl)-2,3-dihydro-1H-indene (S16). An analytical sample of S16 was prepared by flash chromatography (SiO2, 50:1 hexanes/Et₂O): 1H NMR (600 MHz, CDCl3) δH 7.35 (d, J=7.4 Hz, 1H), 7.28-7.20 (m, 3H), 3.82 (d, J=10.2 Hz, 2H), 3.70 (d, J=10.2 Hz, 2H), 2.95 (t, J=7.3 Hz, 2H), 2.27 (t, J=7.3 Hz, 2H); 13C NMR (150 MHz, CDCl3) δ_{C} C: 144.1, 143.6, 52.7; CH: 128.6, 126.5, 125.3, 124.6; CH2: 40.4, 36.3, 29.7; HRMS-DART (m/z) [M+H]+ calculated for C11H13Br₂=302.9384; found 302.9400.

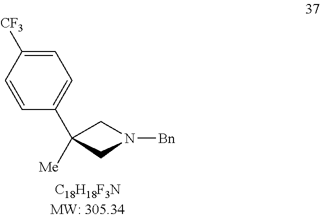

1-benzyl-3-methyl-3-(4-(trifluoromethyl)phenyl)azetidine (37). Following General Procedure C, the title compound was prepared from S7 (112 mg, 0.600 mmol). Purification by flash chromatography (SiO2, 3:1 hexanes/EtOAc) afforded 37 (101 mg, 0.331 mmol, 55% yield) as a colorless oil: 1H NMR (600 MHz, CDCl3) δH7.57 (d, J=7.8 Hz, 2H), 7.32-7.25 (m, 7H), 3.67 (s, 2H), 3.49 (br. s, 2H), 3.39 (br. s, 2H) 1.65 (s, 3H); 13C NMR (150 MHz, CDCl3) δ_{C} C: 153.1, 138.3, 128.3 (q, J=31.9 Hz), 124.4 (q, J=271.3 Hz), 39.3; CH: 128.4, 128.4, 127.2, 125.7, 125.5 (q, J=3.6 Hz); CH2: 65.9, 63.6; CH3: 29.1; 19F NMR (564 MHz, CDCl3) δ_{F} −62.3 (s, 3F); HRMS-DART (m/z) [M+H]+ calculated for C18H19F₃N=306.1470; found 306.1460.

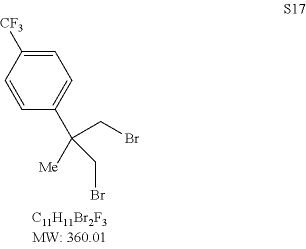 [SMILES: CC(CBr)(CBr)c1ccc(C(F)(F)F)cc1]

1-(1,3-dibromo-2-methylpropan-2-yl)-4-(trifluoromethyl)benzene (S17). An analytical sample of S17 was prepared by flash chromatography (SiO2, 50:1 hexanes/Et₂O): 1H NMR (600 MHz, CDCl3) δH 7.64 (d, J=8.3 Hz, 2H), 7.47 (d, J=8.2 Hz, 2H), 3.78 (d, J=1.7 Hz, 4H), 1.64 (s, 3H); 13C NMR (150 MHz, CDCl3) δ_{C} C: 146.1, 129.7 (q, J=32.1 Hz), 124.1 (q, J=269.8 Hz), 43.4; CH: 126.9, 125.6 (q, J=3.3 Hz); CH2: 41.6; CH3: 24.4; 19F NMR (564 MHz, CDCl3) δ_{F} −62.5 (s, 3F); HRMS-DART (m/z) [M+H]+ calculated for C11H12Br₂F₃=358.9258; found 358.9265.

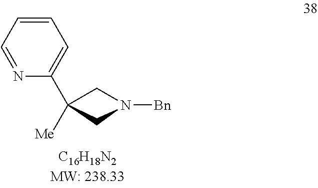

2-(1-benzyl-3-methylazetidin-3-yl)pyridine (38). Following a modification of General Procedure C, a solution of 3 (2.26 mL, 1.20 mmol, 0.53 M in PhMe) was added via syringe to a Schlenk tube and concentrated under reduced pressure (0.1 mBar, 0.5 h). The flask was backfilled with N₂ and the resulting red residue was cooled to −10° C. After 10 min, a pre-cooled solution of S8 (72 mg, 0.60 mmol) in THF (2.0 mL) at 0° C. was added stream-wise via syringe. The resulting red solution was maintained at −10° C. for 3 h, then cooled to −78° C. After 0.25 h, a pre-cooled solution of Br₂ (959 mg, 6.00 mmol) in CH₂Cl₂ (6.0 mL) at −78° C. was added dropwise via syringe. The resulting slurry was maintained at −78° C. for 0.25 h, then warmed to 0° C. After 0.75 h, the reaction septa was removed and the mixture was treated successively with saturated aq. Na₂S₂O₃ (0.35 mL) and trifluoroacetic acid (0.70 mL). The resulting stirred slurry was allowed to warm to r.t. After 3 h, the mixture was treated with saturated aq. NaHCO₃ (6 mL) and stirred at r.t. After 4 h, the reaction mixture was transferred to a separatory funnel and extracted with Et₂O (3×10 mL). The combined organic extracts were dried over MgSO₄, filtered, and concentrated to ˜25% volume under reduced pressure. The resulting slurry was filtered over a pad of SiO₂ (6.5 g/mmol, eluted with 90 mL/mmol of 5:1 hex/EtOAc) and the resulting solution was concentrated under reduced pressure. The resulting crude residue was digested with DMF (2 mL) and treated with sodium iodide (180 mg, 1.20 mmol), potassium carbonate (414 mg, 3.00 mmol), and benzyl amine (642 mg, 6.00 mmol). The resulting slurry was heated to 100° C. After the 50 h, the reaction mixture was cooled to r.t., diluted with Et₂O (5 mL) and filtered. The resulting filtrate was concentrated under reduced pressure and the resulting crude reside was purified by flash chromatography (SiO2, 3:1 hexanes/EtOAc) afforded 38 (149 mg, 0.509 mmol, 85% yield) as a colorless liquid: 1H NMR (600 MHz, CDCl3) δH 8.55 (d, J=4.4 Hz, 1H), 7.63 (td, J=7.7, 1.80, 1H), 7.31-7.25 (m, 4H), 7.24-7.23 (m, 1H), 7.17 (d, J=7.9 Hz, 1H), 7.11 (dd, J=7.9 Hz, 1H), 3.68 (s, 2H), 3.56 (d, J=7.1 Hz, 2H), 3.45 (d, J=7.2 Hz, 2H), 1.68 (s, 3H); 13C NMR (150 MHz, CDCl3) δ_{C} C: 166.9, 138.4, 41.0; CH: 149.2, 136.6, 128.6, 128.4, 127.1, 121.1, 119.8; CH2: 65.2, 63.6; CH2: 27.5; HRMS-DART (m/z) [M+H]+ calculated for C16H19N₂=239.1548; found 239.1545.

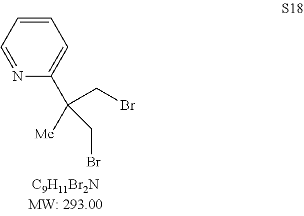 [SMILES: CC(CBr)(CBr)c1ccccn1]

2-(1,3-dibromo-2-methylpropan-2-yl)pyridine (S18). An analytical sample of S18 was prepared by flash chromatography (SiO2, 20:1 hexanes/Et₂O): 1H NMR (600 MHz, CDCl3) δH 9.08 (d, J=5.2 Hz, 1H), 8.26 (t, J=7.9 Hz, 1H), 7.76-7.71 (m, 2H), 4.11 (d, J=10.9 Hz, 2H), 3.79 (d, J=10.9 Hz, 2H), 1.82 (s, 3H). All other characterization was identical to reported values (Siebert, M., et al., J. Am. Chem. Soc. 2019, 141, 334-341).

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches.

1. A process for preparing a compound of Formula I 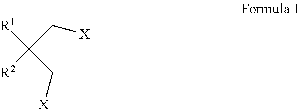
comprising:
(a) treating a compound of formula 10 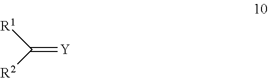
with a compound of formula 11 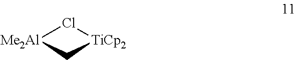
to form a first intermediate and treating the first intermediate with M₂ to form the compound of Formula I;
wherein:
X is a halogen;
Y is O or CH₂;
M is Cl, Br, or I; and
R¹ and R² are each independently selected from hydrogen, substituted or unsubstituted alkyl, cycloalkyl, alkoxy, aryl or heteroaryl, alkylaryl, ether, or R¹ and R², taken together with the atom to which they are directly attached form a four or more membered ring; and
at least one of R¹ or R² are substituted or unsubstituted alkyl, cycloalkyl, alkoxy, aryl or heteroaryl, alkylaryl, or ether.

2. The process of claim 1, wherein R¹ is substituted or unsubstituted aryl.

3. The process of claim 1, wherein R¹ is substituted or unsubstituted alkylaryl.

4. The process of claim 1, wherein R² is substituted or unsubstituted hydrogen, alkyl, cycloalkyl, or alkoxy.

5. The process of claim 1, wherein R¹ and R², taken together with the atom to which they are directly attached form a four or more membered ring.

6. The process of claim 5, wherein R¹ and R², taken together with the atom to which they are directly attached form the ring 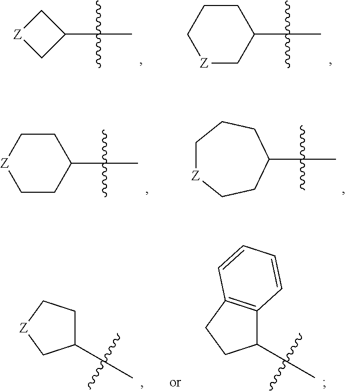
wherein
Z is O, CR⁶R⁷, or NR⁸;
R⁶ and R⁷ are each independently H, or halogen; and
R⁸ is a protecting group.

7. The process of claim 5, wherein R¹ and R², taken together with the atom to which they are directly attached form the ring

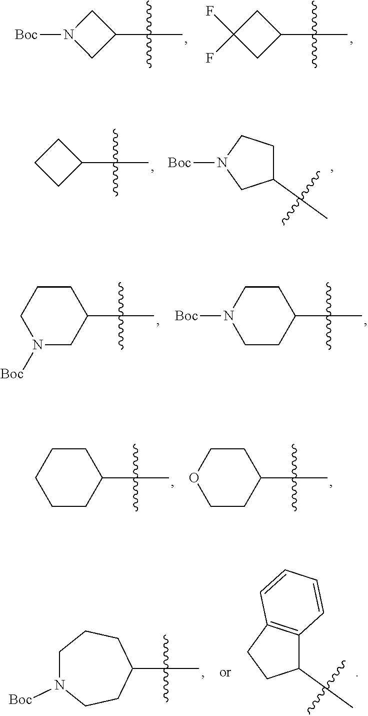

8. The process of claim 1, wherein Y is O.

9. The process of claim 1, wherein Y is CH₂.

10. The process of claim 1, wherein the first intermediate is defined by the structure below:

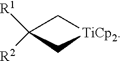

11. The process of claim 1, wherein the process is performed in a solvent.

12. (canceled)

13. The process of claim 11, wherein the solvent comprises acetone, DMSO, DMF, THF, dichloromethane, acetonitrile, or any combination thereof.

14. The process of claim 1, wherein the treating step is performed at 0° C.

15. The process of claim 1, wherein the compound of formula 10 is selected from:

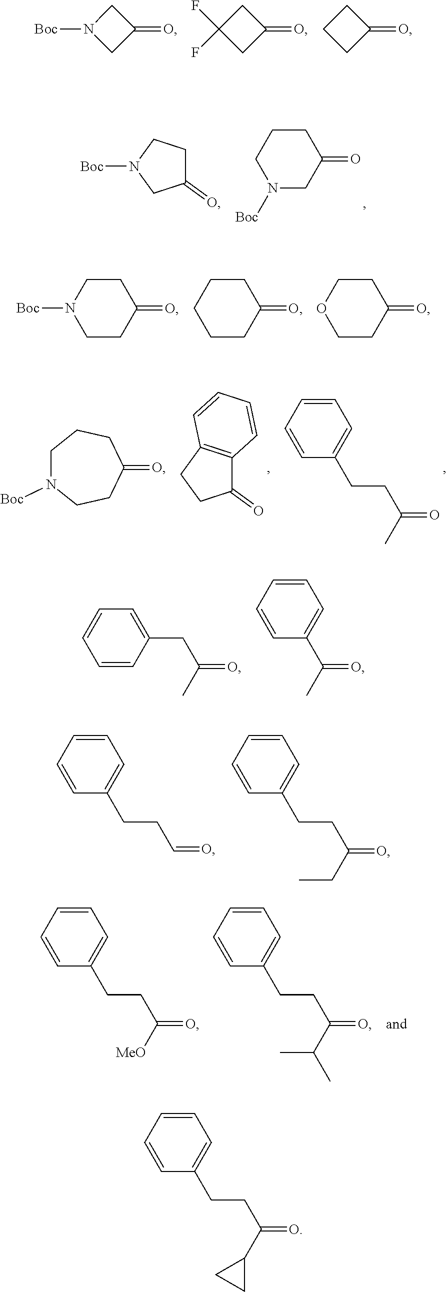

16. The process of claim 1, wherein the compound of Formula I is selected from:

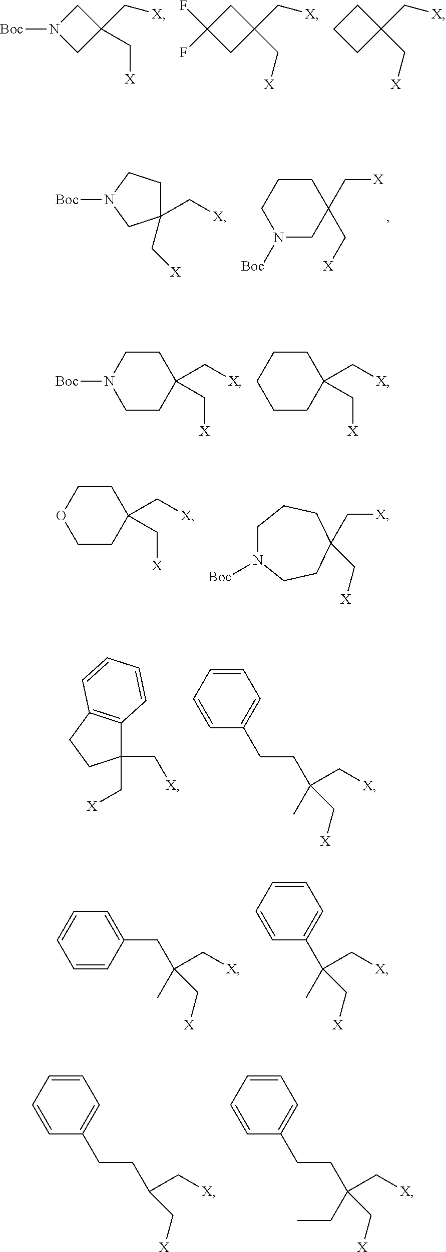

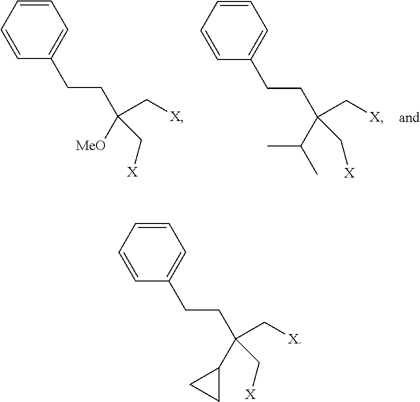

17. (canceled)

18. A process for preparing a compound of Formula IIa 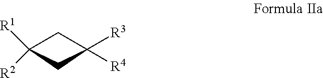
comprising:
(a) treating a compound of Formula I 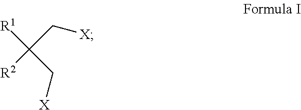
with 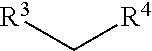
in the presence of a base to form a compound of Formula IIa;
wherein:
X is a halogen;
R¹ and R² are each independently selected from hydrogen, substituted or unsubstituted alkyl, cycloalkyl, alkoxy, aryl or heteroaryl, alkylaryl, ether, or R¹ and R², taken together with the atom to which they are directly attached form a four or more membered ring; and at least one of R¹ or R² are substituted or unsubstituted alkyl, cycloalkyl, alkoxy, aryl or heteroaryl, alkylaryl, or ether; and
R³ and R⁴ are ester.

19. (canceled)

20. (canceled)

21. A process for preparing a compound of Formula IIb 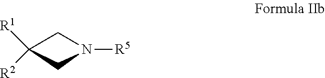
comprising:
(a) treating a compound of Formula I 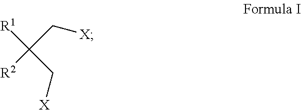
with R⁵—NH₂ in the presence of a base to form a compound of Formula IIb;
wherein:
X is a halogen;
R¹ and R² are each independently selected from hydrogen, substituted or unsubstituted alkyl, cycloalkyl, alkoxy, aryl or heteroaryl, alkylaryl, ether, or R¹ and R², taken together with the atom to which they are directly attached form a four or more membered ring; and at least one of R¹ or R² are substituted or unsubstituted alkyl, cycloalkyl, alkoxy, aryl or heteroaryl, alkylaryl, or ether; and
R⁵ is substituted or unsubstituted alkenyl, alkylaryl, aryl, or heteroaryl.

22-49. (canceled)

50. The process of claim 18, wherein the compound of Formula I is selected from:

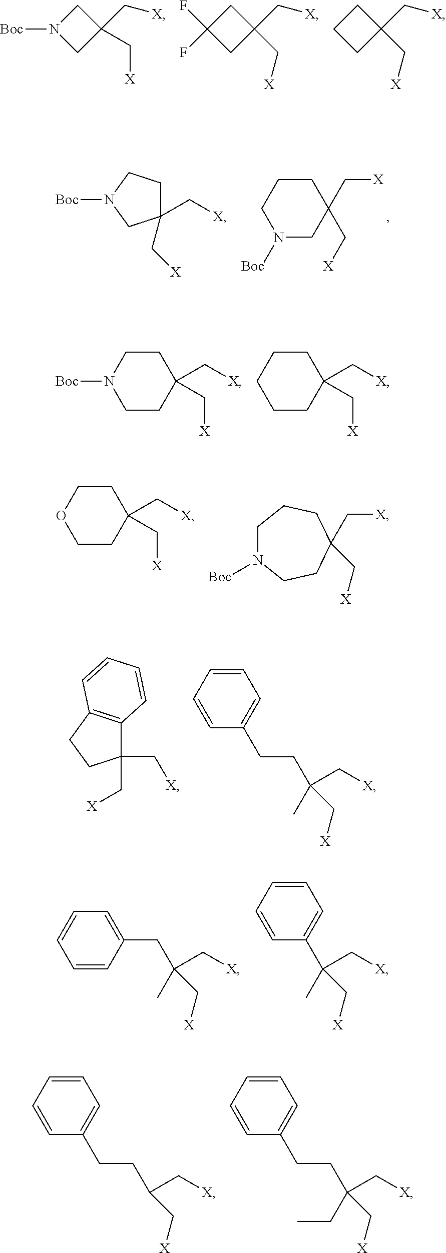

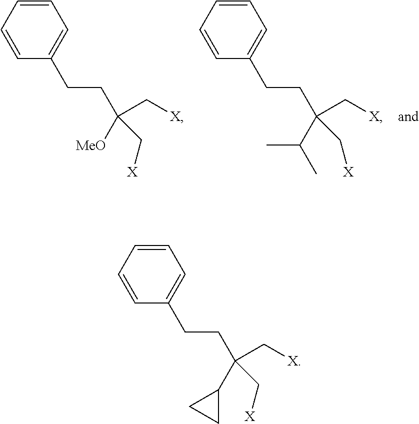

51. The method of claim 18, wherein the compound of Formula IIa, IIb, IIc, or IId is selected from: 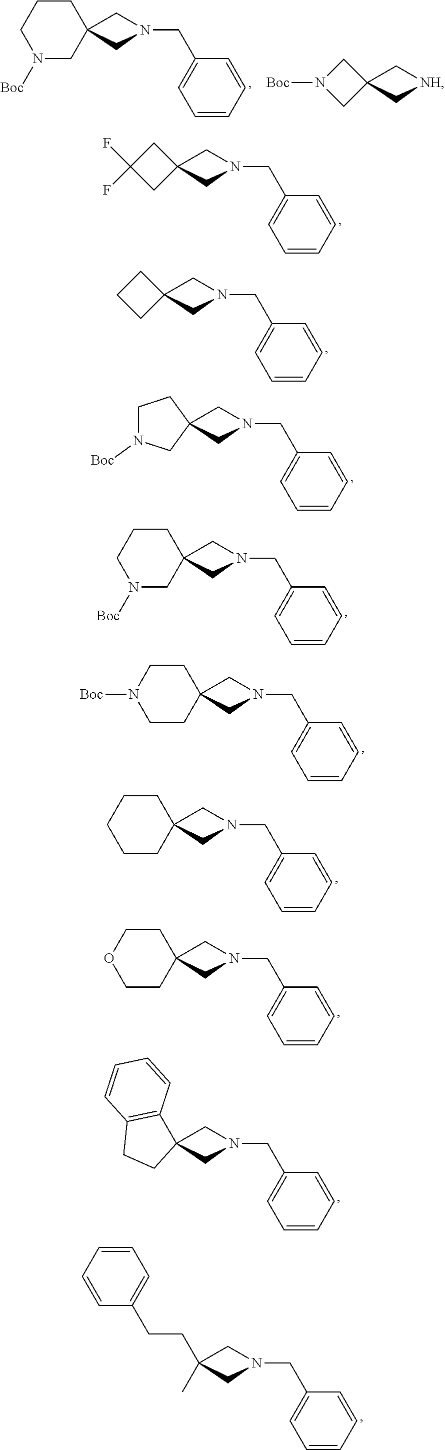 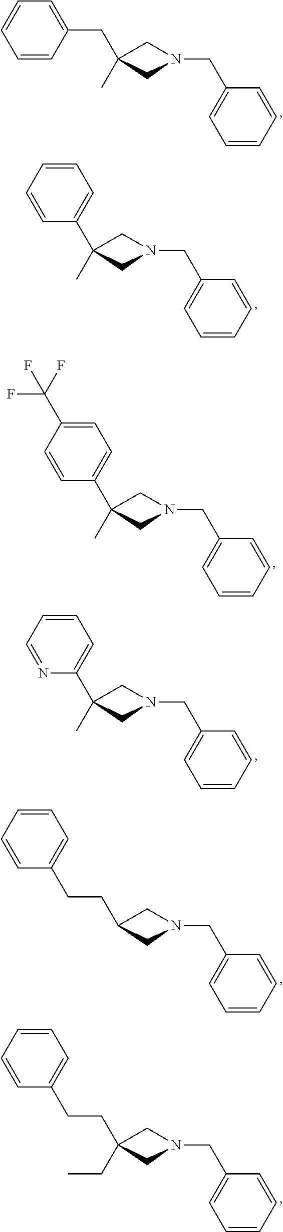 [chemical structure]
